(19) United States
(12) Patent Application Publication
(10) Pub. No.: US 2021/0028324 A1
(43) Pub. Date: Jan. 28, 2021

(54) BACTERIAL CELLULOSE PAPER-BASED FLEXIBLE ELECTRONICS EMPLOYING NANOCRYSTALS

(71) Applicant: Board of Regents, The University of Texas System, Austin, TX (US)

(72) Inventors: Brian A. Korgel, Austin, TX (US); Vikas Reddy Voggu, Austin, TX (US); James Sham, Austin, TX (US); R. Malcolm Brown, Austin, TX (US); Taylor B. Harvey, Austin, TX (US)

(73) Assignee: Board of Regents, The University of Texas System, Austin, TX (US)

(21) Appl. No.: 17/060,930

(22) Filed: Oct. 1, 2020

Related U.S. Application Data
(62) Division of application No. 16/337,240, filed on Mar. 27, 2019.
(60) Provisional application No. 62/400,209, filed on Sep. 27, 2016.

(51) Int. Cl. H01L 31/0392; C12P 19/04; D21H 13/02; D21H 15/02; H01L 23/14; H01L 23/498; H01L 31/02; H01L 31/0224; H01L 31/0296; H01L 31/032; H01L 31/0368; H01L 31/068; H01L 31/0749; H01L 31/18; H01L 51/00; H01L 31/0236; C08L 1/02; B82Y 40/00; D21H 11/18

(52) U.S. Cl. CPC H01L 31/03928; B82Y 30/00; D21H 13/02; D21H 15/02; H01L 23/145; H01L 23/4985; H01L 31/02008; H01L 31/022425; H01L 31/0296; H01L 31/0322; H01L 31/0368; H01L 31/068; H01L 31/0749; H01L 31/1836; H01L 51/0097; H01L 31/03926; H01L 31/02366; C08L 1/02; B82Y 40/00; D21H 11/18; H01L 31/022475; Y02E 10/541; Y02E 10/549; C12P 19/04

(57) ABSTRACT

Described are flexible electronics incorporating a bacterial cellulose paper substrate and methods of making and using the flexible electronics. Example devices disclosed include photovoltaic cells constructed over bacterial cellulose paper substrates.

### CROSS REFERENCE TO RELATED APPLICATION

This application is a divisional of U.S. application Ser. No. 16/337,240, filed on Mar. 27, 2019. U.S. application Ser. No. 16/337,240 is the national stage of PCT application PCT/US2017/053673 filed on Sep. 27, 2017, which claims the benefit of and priority to U.S. Provisional Application 62/400,209, filed on Sep. 27, 2016. All of the foregoing applications are hereby incorporated by reference in their entireties.

### BACKGROUND

This invention is in the field of micro- and nano-scale electronic devices. This invention relates generally to electronic devices fabricated on flexible cellulose substrates.

The global impact of paper throughout history cannot be overstated. As an innovation, paper not only precedes the advent of written history, its invention enabled the entire practice. Furthermore as a commodity, paper and paper products have a presence in nearly every sector of the global economy, accompanying or comprising many of society's consumables. To meet the demand nearly 400 million tonnes of paper is produced annually. That said, paper is not yet used as a substrate to build electronics on a commercial scale.

### SUMMARY

Described are flexible electronics incorporating a bacterial cellulose paper substrate and methods of making and using the flexible electronics. Example devices disclosed include photovoltaic cells constructed over bacterial cellulose paper substrates.

In a first aspect, flexible electronic devices are provided. In some embodiments, a flexible electronic device of this aspect comprises a flexible substrate comprising paper including cellulose nano-fibers having average diameters between about 50 nm and about 150 nm; and a flexible electronic device component supported by the flexible substrate. For example, in some embodiments, the flexible electronic device component comprises a crystalline inorganic semiconductor material. In some embodiments, the flexible substrate comprises bacterial cellulose paper.

In another aspect, methods of making flexible electronic devices are provided. In some embodiments, a method of this aspect comprises providing a flexible substrate comprising paper including cellulose nano-fibers having average diameters between about 50 nm and about 150 nm; and depositing one or more flexible device components over the substrate. For example, in some embodiments, the one or more flexible device components include at least a crystalline inorganic semiconductor material. In some embodiments, the crystalline inorganic semiconductor material is deposited over the flexible substrate using a solution processing method.

In another aspect, methods of using flexible electronic devices are provided. In some embodiments, a method of this aspect comprises providing the flexible electronic device, such as a flexible electronic device that comprises: a flexible substrate comprising bacterial cellulose paper; and a flexible electronic device component supported by the flexible substrate, such as a flexible electronic device component that comprises a crystalline inorganic semiconductor material; and providing current or voltage for use by the flexible electronic device component, or generating current or voltage using the flexible electronic device component, or generating current or voltage using a first portion of the electronic device component for use by a second portion of the electronic device component.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1A, FIG. 1B, and FIG. 1C provide schematic illustrations of a flexible electronic device.

FIG. 2A and FIG. 2B provide schematic illustrations of nanocellulose paper and regular paper, showing individual cellulose fibers.

FIG. 3 provides an overview of a method of making bacterial cellulose paper.

FIG. 4 provides an overview of a method of making a flexible electronic device.

FIG. 5 provides scanning electron micrograph (SEM) images of various kinds of paper.

FIG. 6 provides data showing the effect of layer thickness on device performance and images of various devices.

FIG. 7 provides a JV curve of one flexible photovoltaic device and images of various devices and plain bacterial cellulose paper.

FIG. 8 provides a plot of average device characteristics as a function of flex cycles for different devices, JV curves a flexible photovoltaic device in flat orientation after bending to various radii, and images of various devices.

FIG. 9 provides schematic illustrations showing the difference in the measurement in flat configuration vs curved configuration devices, JV curves of flexible photovoltaic cells while being bent to different radii, and device characteristics flexible photovoltaic devices plotted as a function of bending radii.

FIG. 10 provides a JV curve for a flexible device on bacterial cellulose before and after folding, with the inset showing the voltage being measured in ambient room lighting with a voltmeter and SEM images different devices.

FIG. 11 provides: schematics showing different layers of a solar cell and images of a formed solar cell in use.

FIG. 12 provides images of different kinds of paper after various deposition processes.

FIG. 13 provides plots of power conversion efficiency (PCE) for CuInSe₂ (CIS) nanocrystal (NC) photovoltaics (PVs) on bacterial cellulose before ( [symbol] ) and after ( [symbol] ) baking at 200° C. in vacuum and holding for (panel a) 0 minutes, (panel b) 5 minutes, and (panel c) 10 minutes.

FIG. 14 provides a plot of device characteristics as a function of flex cycles for four CIS NC PVs on bacterial cellulose to a radius of 2 mm.

### DETAILED DESCRIPTION

### I. General

The present disclosure relates generally to electronic devices constructed on paper. More specifically, flexible electronic devices are described in which the devices are supported by a flexible paper substrate including cellulose nano-fibers having average diameters between about 50 nm and 150 nm. Example average diameters may be 50 nm, 55, nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. Average diameters may fall in any range between about 50 nm and 150 nm, such as between 50 nm and 60 nm, between 60 nm and 70 nm, between 70 nm and 80 nm, between 80 nm and 90 nm, between 90 nm and 100 nm, between 100 nm and 110 nm, between 110 nm and 120 nm, between 120 nm and 130 nm, between 130 nm and 140 nm, or between 140 nm and 150 nm. The flexible substrate of the disclosed devices may correspond to paper comprising bacterial cellulose, which may be obtained from a culture of a suitable bacteria and processed to form a paper substrate. These devices may exhibit significant flexibility and bending ability, including the ability to bend or flex to a radius of curvature as small as about 3 mm, such as between 3 mm and 5 mm, between 3 mm and 6 mm, between 3 mm and 7 mm, between 3 mm and 8 mm, between 3 mm and 10 mm, between 3 mm and 15 mm, between 3 mm and 20 mm, between 3 mm and 25 mm, between 3 mm and 50 mm, or between 3 mm and 100 mm. Moreover, the flexible cellulose nano-fiber substrates may, in some embodiments, be stretchable in addition to flexible. For example, in some embodiments, the electronic devices may be stretched to 100-125% or more of an original lateral dimension. This stretching ability may provide for a robust device that can withstand a variety of stresses and strains, both compressive and expansive, allowing for use of the electronic devices in a variety of applications. Example applications for the flexible devices include use in a label, a sticker, a sensor, a body-integrated device, a drone, a photovoltaic tape, a photovoltaic wallpaper, a photovoltaic window covering, a man-made structure, or an electronic display.

Advantageously, the disclosed devices incorporate flexible electronic device components supported by the flexible substrate, such as a flexible device component that comprises a crystalline inorganic semiconductor material. It will be appreciated that crystalline inorganic semiconductors may exhibit advantages over organic materials, such as organic semiconductors and organic dyes commonly utilized in organic photovoltaics, dye-sensitized solar cells, and organic light emitting diodes. For example, crystalline inorganic semiconductors may exhibit better thermal stability, better air stability, higher carrier mobilities, and improved longevity as compared to organic-based devices. Using crystalline inorganic semiconductor materials, a variety of different device types may be supported by the flexible substrate, including semiconductor junctions, light emitting diodes, thin film transistors, etc.

Disclosed flexible devices may incorporate a paper substrate-based photovoltaic cell, which may provide the ability to generate power at the point of use. Additionally, the photovoltaic cells described are able to be fabricated without use of a high-vacuum or cleanroom level fabrication facility, and may be created in a simple wet laboratory environment at the point of use, minimizing freight, transportation, and associated costs typically encountered with conventional photovoltaic cells constructed on glass or other rigid substrates.

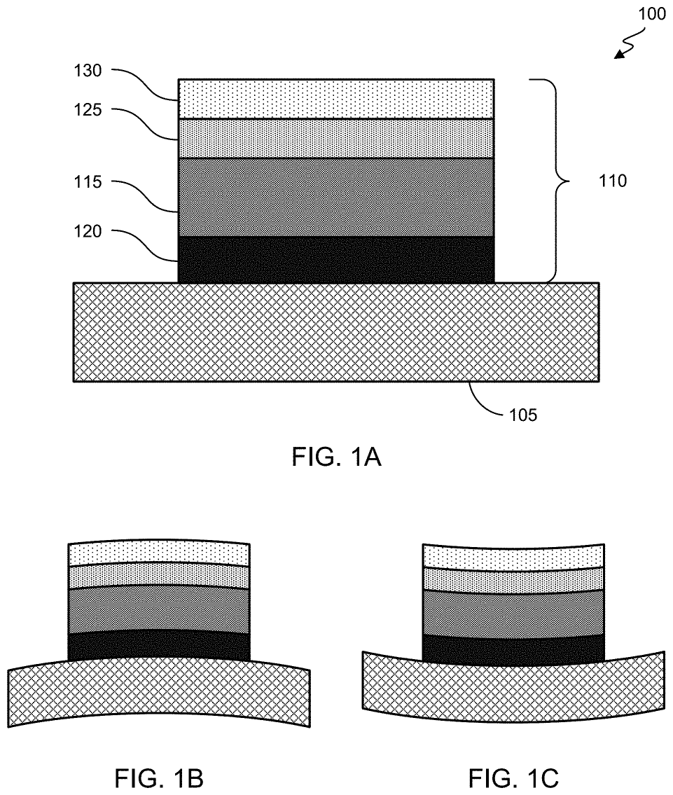

FIG. 1A provides a schematic illustration of an embodiment of a flexible electronic device 100. Flexible electronic device 100 includes a flexible substrate 105 comprising paper, such as including cellulose nano-fibers having average diameters between about 50 nm and about 100 nm. In FIG. 1A, flexible electronic device 100 corresponds to a flexible photovoltaic device.

A flexible device component 110 is supported by flexible substrate 105. Flexible device component 110 includes a crystalline semiconductor material 115, in addition to other elements. The other elements of flexible device component 110 include conductor 120, semiconductor 125, and transparent conductor 130. It will be appreciated that each layer may comprise multiple sub-layers.

As a specific example, substrate 105 may comprise bacterial cellulose paper, conductor 120 may comprise gold, crystalline semiconductor material 115 may comprise CuInSe₂, semiconductor 125 may comprise a CdS layer and a ZnO layer, and transparent conductor 130 may comprise indium tin oxide (ITO).

FIG. 1B illustrates flexible electronic device 100 bent to a convex curvature and FIG. 1C illustrates flexible electronic device 100 bent to a concave curvature. It will be appreciated that the curvatures illustrated are merely examples, and that any complex curvature, including both convex and concave regions, are compatible with the disclosed devices. Additionally, for some embodiments, the devices may be folded, such as where the flexible substrate is pressed into a creased form that is retained. It will be further appreciated that the devices may be unfolded and the creases smoothed to return to a more planar configuration.

### II. Definitions

In general the terms and phrases used herein have their art-recognized meaning, which can be found by reference to standard texts, journal references and contexts known to those skilled in the art. The following definitions are provided to clarify their specific use in the context of the invention.

“Cellulose” refers to a polymer or polysaccharide comprising a chain of glucose molecules, such as D-glucose. Chains or strands of cellulose may agglomerate to form larger cellulose fibers, and individual fibers may further agglomerate to form even larger fibers. Cellulose fibers may be joined in a web or matrix to form various types of paper, which may also include other impurities, such as water, hemicellulose, lignin, pectin, trace elements, dissolved salts, etc.

“Bacterial cellulose” refers to cellulose generated by a bacterium and is contrasted with cellulose generated by a plant cell. Bacterial cellulose may take the form of fibers of pure or crystalline cellulose molecules, which may contain low or no amounts of impurities, such as less than about 10% by weight, less than about 5% by weight, less than about 1% by weight, or less than about 0.1% by weight, and which may exhibit fiber diameters of about 50 nm to about 150 nm, in some embodiments. Bacterial cellulose may correspond to a pure or substantially pure cellulose pulp. In some embodiments, bacterial cellulose may also encompass cellulose generated by other single celled organisms that produce crystalline nanocellulose, such as algae. In some embodiments, bacterial cellulose is generated by bacteria of the genus Gluconobacter or Acetobacter. In some embodiments, bacterial cellulose is generated by bacteria of the species Gluconobacter hansenii or Acetobacter xylinium.

“Bacterial cellulose paper” refers to a matrix or web of bacterial cellulose formed into a thin sheet. Bacterial cellulose paper useful with some embodiments described herein may correspond to a sheet or web comprising cellulose fibers, such as crystalline cellulose fibers, in which the cellulose fibers have an average diameter of about 50 nm to about 150 nm. It will be appreciated that the average diameter of the cellulose fibers may be determined by, for example, obtaining a micrograph or microscope image, identifying the diameters of the cellulose fibers, and taking the arithmetic mean of the observed fiber diameters. Average fiber length may be similarly determined. Bacterial cellulose paper may consist of or consist essentially of cellulose fibers, typically without additives, though some bacterial cellulose paper may include dopants or impurities. Unlike paper made from plant cellulose, bacterial cellulose paper may not require processing to remove nonaqueous impurities, like lignin, to generate cellulose pulp. The cellulose fibers made may also have lengths comparable to cellulose fibers made by plants and may, in some embodiments, have lengths longer than plant made cellulose fibers or lengths shorter than plant made cellulose fibers. In some embodiments, the cellulose fibers may have average lengths of several microns to several millimeters. Bacterial cellulose paper useful with some aspects described herein may correspond to a sheet or web comprising cellulose fibers that exhibits a surface roughness of about 30 nm to about 120 nm or about 50 nm to about 100 nm. In some embodiments, a surface roughness of a bacterial cellulose paper may be attributable to a size of the cellulose fibers comprising the bacterial cellulose paper.

“Flexible” refers to a characteristic of a material to undergo bending or elastic deformation and return to its original shape when an applied stress is removed. In some embodiments, flexible material may be bent or elastically deformed without sustaining substantial damage, such as damage characteristic of destruction or separation of substantial portions of the material. In some embodiments, the term “flexible” and the term “bendable” are used interchangeably herein. In some embodiments, flexible materials may undergo bending to a radius of curvature of 3 mm or greater, such as between 3 mm and 5 mm, between 3 mm and 6 mm, between 3 mm and 7 mm, between 3 mm and 8 mm, between 3 mm and 10 mm, between 3 mm and 15 mm, between 3 mm and 20 mm, between 3 mm and 25 mm, between 3 mm and 50 mm, or between 3 mm and 100 mm, without cracking, fracturing, or otherwise sustaining damage characteristic of failure, destruction, or inelastic deformation. In some embodiments, electronic devices may be flexible, which may indicate that the devices can be deformed to some degree without sustaining damage characteristic of failure, destruction, or inelastic deformation of the device. In some embodiments, flexible materials may also be stretchable. Materials may be flexed or bent by exposing the material to a suitable force, a suitable stress, or a suitable strain, for example. Example elastic moduli for flexible materials include those between 0.01 GPa and 3.0 GPa.

“Stretchable” refers to a characteristic of a material to undergo reversible elongation and return to its original shape when an applied stress is removed. In some embodiments, stretchable material may be elastically elongated without sustaining substantial damage, such as damage characteristic of destruction or separation of substantial portions of the material. Materials may be stretched by exposing the material to a suitable force, a suitable stress, or a suitable strain, for example.

“Young's modulus” and “elastic modulus” refer to a mechanical property of elastic solid materials that defines the relationship between stress and strain in the material. In an embodiment, the Young's modulus of a material is computed by dividing the stress by the strain. The Young's modulus of a material may alternatively be computed by dividing the product of an applied force and an original length of an object by the product of the cross sectional area through which the force is applied and the change in length of the object. Many materials have an intrinsic Young's modulus that may, in embodiments, relate to how stiff or flexible the material is. For example, materials with a lower Young's modulus may be more flexible or stretchable than materials with a higher Young's modulus.

“Substrate” refers to a material having a surface that is capable of supporting other components, such as an electronic device component. In some embodiments, a substrate may have a thickness less or about equal to that of a supported component. In some embodiments, a substrate may have a thickness greater than or about equal to that of a supported component. In some embodiments, a substrate may be flexible or stretchable, such as a substrate comprising paper, for example bacterial cellulose paper.

“Crystalline” refers to a characteristic of a material whose components are arranged in a highly ordered microscopic structure, where the structural elements align in a lattice or repeating structure. In some embodiments, crystalline materials may take on single crystal forms or polycrystal forms.

“Nanocrystalline” refers to a crystalline nanoparticle form of a material in the individual nanoparticles exhibit at least one dimension having a size smaller than about 100 nm. Nanocrystalline materials may be single crystal or polycrystalline materials.

“Semiconductor” refers to a material that exhibits conductivity characteristics between a metal and an insulator. Semiconductors exhibit a band gap between a conduction band and a valence band such that the band gap may be small enough that for thermal or electrical effects to impact the semiconductor transitioning between an insulating material and a conducting material. For example, in some embodiments, a semiconductor is an insulator at or below a particular temperature and may exhibit a substantial electrical conductivity, approaching that of a conductive metal, as the temperature is increased. Semiconductors may be characterized as n-type or p-type. For example, in n-type semiconductors, thermal effects may allow electron populations to reach the conduction band, while in p-type semiconductors, electron populations may be limited to the valence band. Use of the term semiconductor is intended to be consistent with use of this term in the art of microelectronics and electrical devices. Useful semiconductors include element semiconductors, such as silicon, germanium, and diamond, compound semiconductors, group IV compound semiconductors (e.g., SiC and SiGe), group III-V semiconductors (e.g., AlSb, AlAs, Aln, AlP, BN, GaSb, GaAs, GaN, GaP, InSb, InAs, InN, and InP), group III-V ternary semiconductors alloys (e.g., AlₓGa₁₋ₓAs), group II-VI semiconductors (e.g., CsSe, CdS, CdTe, ZnO, ZnSe, ZnS, and ZnTe), group I-VII semiconductors (e.g., CuCl), group IV-VI semiconductors (e.g., PbS, PbTe, and SnS), layer semiconductors (e.g., PbI₂, MoS₂, and GaSe), oxide semiconductors (e.g., CuO and Cu₂O), I-III-VI₂ chalcopyrite compound semiconductors (e.g., CuInSe₂, CuGaS₂, and CuInTe₂), and mixtures thereof.

“Surface roughness” refers to a measure of the deviations in a surface from a perfectly planar surface. In some embodiments, surface roughness may characterize the size of the deviations in the surface, which may relate to the size of the material that makes up the surface. For example, in a surface comprising a web or matrix of nanofibers, the diameter dimension of the nanofibers may relate to the roughness of the surface. It will be appreciated that, in some embodiments, a surface roughness may correspond to a root mean squared roughness value, which may be computed as the square root of an arithmetic mean of the squares of a set of deviations from a mean surface position.

“Lateral” refers to a direction that may be orthogonal to a particular axis or dimension of a material, such as a thickness direction.

“Damage associated with impaired device performance” refers to a structural defect, such as a crack, fissure, or fracture, that results in a device or device component being rendered inoperable or having reduced performance. For example, in some embodiments, a crack in a conductive material may cause the material to no longer be useful for carrying electrons from a first point on one side of the crack to a second point on the other side of a crack. Materials that are damaged may also exhibit reduced performance, such as an increase in electrical resistivity, due to the fracture or cracking of the material, such as where electrons can no longer efficiently be transported between locations in the material. In some embodiments, damage associated with impaired device performance may be transitory and may be repaired or relieved by changing a material geometry, such as changing from a bent or stretched formation to a relaxed formation. For example, in some embodiments, an electronic device supported by a paper substrate may exhibit damage associated with impaired device performance when the in a folded configuration, and when in an unfolded configuration, the electronic device may return to optimal or near optimal performance.

“Nanowires” refers to a conducting or semiconducting material in which the material is in the form of long rod-like filaments that exhibit a diameter of about 100 nm. In some embodiments, nanowires exhibit diameters of about 10 nm to about 25 nm. Length to diameter ratios of nanowires may be in the range of about 100 to about 1000, or more, in some embodiments.

“Nanofibers” refers to a material having a long, thin, and rod-like configuration in which the average diameter may be between about 1 nm and about 500 nm. In some embodiments, nanofibers may have an average diameter of about 100 nm. In some embodiments, nanofibers comprise a polymeric material, such as a polymer chain, which may contribute to the structure and strength of the nanofiber. Cellulose nanofibers useful with some aspects described herein may correspond to a fiber composed of cellulose molecules, which may exhibit a crystalline structure.

“Conformal contact” and “intimate contact” refers to an arrangement of two components such that the surfaces of the two components maintain contact over an area without voids between the surfaces. Conformal contact may be of particular relevance, in some embodiments, for surfaces that exhibit raised regions or recessed regions. For example, in one embodiment, a first surface that includes raised and recessed regions may be conformally contracted by a second surface that maintains contact with the first surface over the raised and recessed regions without voids between the surfaces at the raised and recessed regions.

### III. Flexible Substrates

As described above, the flexible substrates useful with the flexible electronic devices described herein include those comprising paper, such as paper comprising cellulose nano-fibers having average diameters between about 50 nm and about 150 nm. In some embodiments, the flexible substrate comprises crystalline cellulose. In some embodiments, the flexible substrate comprises cellulose fibers having purities of greater than 90%, for example, such as greater than 91%, greater than 92%, greater than 93%, greater than 94%, greater than 95%, greater than 96%, greater than 97%, greater than 98%, greater than 98%, greater than 99%, greater than 99.5%, or greater than 99.7%. Useful flexible substrates include those having a thickness of about 0.5 μm to about 100 μm, such as thicknesses of 0.5 μm, 1 μm, 5 μm, 10 μm, 20 μm, 50 μm, or 100 μm.

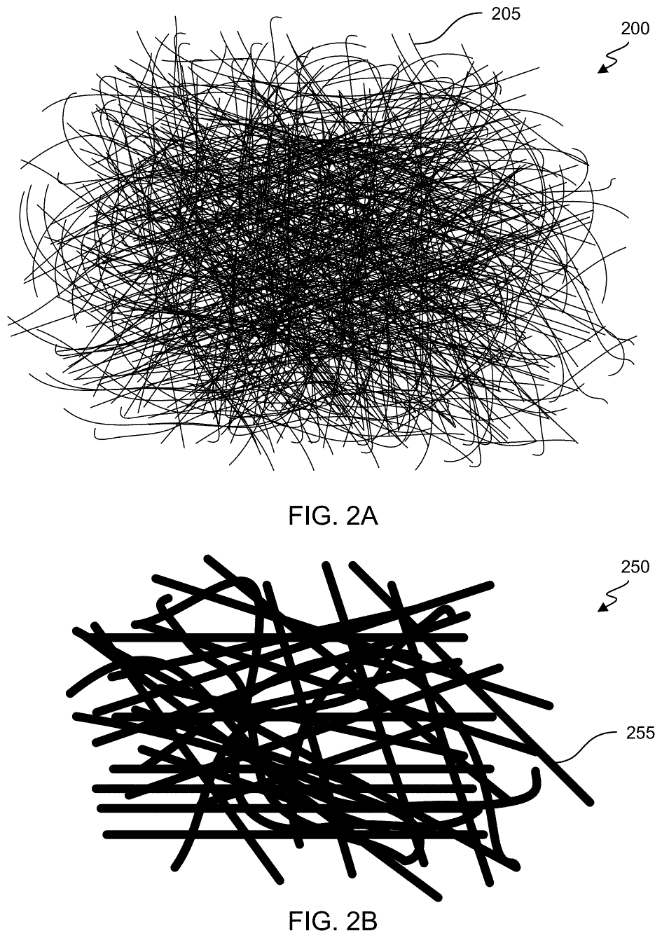

FIG. 2A provides a schematic illustration of a magnified view of a portion of a flexible substrate 200 comprising a plurality of cellulose nano-fibers 205. Here, the nano-fibers are constructed as a web or matrix of overlapping crystalline cellulose fibers with diameters of between about 50 nm and 150 nm.

For contrast, FIG. 2B provides a schematic illustration of a magnified view of a portion of a paper 250 made of conventional cellulose harvested from plant sources. The cellulose fibers 255 of paper 250 are considerably larger than cellulose nano-fibers 205. Additionally, the average inter-fiber spacing of between cellulose fibers 255 is also considerably larger than that of flexible substrate 200.

As an example, bacterial cellulose paper has been identified as a substrate useful with the flexible electronic devices. Moreover, bacterial cellulose paper has a variety of advantageous characteristics. Bacterial cellulose paper may be obtained, for example, by harvesting cellulose nano-fibers generated by a culture of a bacterium of the genus Gluconacetobacter or Acetobacter, such as the bacterial species Gluconacetobacter hansenii or Acetobacter xylinium. It will be appreciated that other bacteria may be capable of generating cellulose nano-fibers, such as certain bacteria species from the genera Azotobacter, Rhizobium, Pseudomonas, Salmonella, and Alcaligenes. Cellulose nano-fibers generated by bacteria may advantageously be crystalline, of high purity, and may comprise cellulose-nano fibers having diameters of about 100 nm, for example.

Bacterial cellulose paper may be advantageously generated in a wet-laboratory by growing a culture of cellulose producing bacteria, harvesting cellulose from the culture, and pressing the cellulose to form a sheet of bacterial cellulose paper. In some embodiments, the bacterial cellulose paper is dried, though in other embodiments the bacterial cellulose paper may be used wet.

Paper comprising cellulose nano-fibers having average diameters between about 50 nm and about 150 nm may exhibit other desirable properties, including, but not limited to, a surface roughness of about 25 nm to about 125 nm, or about 50 nm to about 100 nm. Surface roughness of the substrate may impact the ability of device components to conformally or intimately contact the substrate and form strong bonds with the substrate. Such properties may impact the ability of the device components to flex with the substrate and prevent separation from the substrate. It will be appreciated that when a device component separates, at least partly, from a substrate, the device components may exhibit stresses and strains that may result in damage to the device components, such as cracking, which may impair device performance.

Other advantageous properties of a flexible substrate, such as a cellulose nano-fiber based paper substrate, include its ability to elastically bend to a very small radius of curvature, such as a radius of curvature of about 3 mm. Although cellulose nano-fiber based paper may be bent to a smaller radius of curvature than 3 mm, the inventors have observed that when device components are assembled on a cellulose nano-fiber based paper, the device components can also be elastically bent to a radius of curvature as small as about 3 mm without the device components undergoing damage. In some embodiments, the flexible substrate is bendable to a radius of curvature of about 3 mm or greater, such as between 3 mm and 5 mm, between 3 mm and 6 mm, between 3 mm and 7 mm, between 3 mm and 8 mm, between 3 mm and 10 mm, between 3 mm and 15 mm, between 3 mm and 20 mm, between 3 mm and 25 mm, between 3 mm and 50 mm, or between 3 mm and 100 mm, and subsequently returned to a flat or substantially flat configuration.

In some embodiments, the flexible substrate may also be folded, that is creased such that the flexible substrate holds the creased shape. In some embodiments, the crease may correspond to an inelastic deformation of the flexible substrate. In some embodiments, the crease may correspond to bending to a radius of curvature of less than 3 mm. The inventors have observed that, in some embodiments, devices assembled on a flexible substrate that is subsequently folded may continue to function while folded, while other devices may undergo damage resulting in failure of the device components. In some embodiments, the devices that fail while folded may return to operational states upon unfolding the flexible substrate.

In some embodiments, the flexible substrate may be stretchable. The electronic device may optionally be stretched by exposing the substrate to a stretching force or otherwise elongating the substrate in one or more lateral dimensions. In some embodiments, the flexible substrate is stretchable in a lateral direction to a size between about 100% and about 150% of its original or unstretched size. In some embodiments, the flexible substrate is stretchable in a lateral direction to a size between about 100% and about 105%, between about 100% and about 110%, between about 100% and about 115%, between about 100% and about 120%, or between about 100% and about 125% of its original or unstretched size without the device sustaining damage associated with impaired device performance.

### IV. Electronic Device Components

As noted above, flexible electronic devices described herein may include flexible electronic device components supported by the flexible substrate. Like the flexible substrate, in some embodiments, the electronic device components may themselves be flexible. For example, a flexible electronic device component may optionally undergo bending to a radius of curvature of 3 mm or more without sustaining damage associated with impaired device performance, such as a radius of curvature of between 3 mm and 5 mm, between 3 mm and 6 mm, between 3 mm and 7 mm, between 3 mm and 8 mm, between 3 mm and 10 mm, between 3 mm and 15 mm, between 3 mm and 20 mm, between 3 mm and 25 mm, between 3 mm and 50 mm, or between 3 mm and 100 mm. In some embodiments, the flexible electronic device component is free of cracks or fractures that cause failure or short-circuiting of the electronic device component, even while in a curved or bent configuration.

The ability to bend the electronic device component may arise, at least in part, due to a size dimension of the electronic device component, such as a thickness dimension. The electronic device components may also have any suitable thickness. In some embodiments, the device component has a thickness of about 10 nm to about 1000 nm, or about 10 nm to about 500 nm. In some embodiments, as illustrated in FIGS. 1A, 1B, and 1C, multiple device components are arranged over the flexible substrate, and each device component may have different thicknesses. In some embodiments, an overall, combined total thickness of the flexible electronic device component(s) may be between about 10 nm and 1000 nm, depending on the configuration.

The ability to bend the electronic device component may arise, at least in part, due to contact and adhesion with the underlying material. For example, in some embodiments, the electronic device component is in physical contact with the flexible substrate. Optionally, the electronic device component is indirect physical contact with the flexible substrate, meaning that the electronic device component is in physical contact with another electronic device component that itself is in direct or indirect physical contact with the flexible substrate. Upon bending the flexible substrate, in some embodiments, a force may be imparted to the electronic device component that provides a suitable stress or strain to result in bending or flexing the electronic device component.

The electronic device components may also have any suitable lateral dimensions. For example, in some embodiments, an electronic device component has a lateral dimension as small as 10 nm, as large as 1 μm, or as large as 10 cm or even larger. It will be appreciated that, in some embodiments, the lateral dimensions are only limited by that of the flexible substrate on which the electronic device component is built on. In some embodiments, the lateral dimensions may be dictated by the type of device and the functionality of the device required. Conventional process steps of patterning using masking, lift-off, and the like, may be used to create electronic device components of desirable lateral dimensions.

Useful electronic device components include, but are not limited to, those comprising a semiconductor, such as a crystalline inorganic semiconductor, a conductor, such as a metal, a metal alloy, or a transparent conductor, and an insulator, such as a dielectric. Useful conductors include Cr, Au, Pd, Al, Cu, Pt, Ni, Co, Ag, Mo, indium tin oxide (ITO), and other metals and transparent conductors. Useful dielectrics include non-conducting oxides, photoresists, polymers, such as polyethylene terephthalate (PET), polydimethylsiloxane (PDMS), polyimide, plastics, and the like

### V. Semiconductor Materials

At least one flexible electronic device component may comprise a crystalline inorganic semiconductor material, such as a nanocrystalline material. For example, in some embodiments, the crystalline inorganic semiconductor material may comprise a plurality of nanoparticles or a plurality of nanowires. Any suitable semiconductor may be included as a flexible electronic device component of the flexible electronic device. Use of nanoparticles or nanowires may be advantageous, for some embodiments, as these types of crystalline materials may be deposited onto a substrate using a solution processing method. Example inorganic semiconductor materials include CuInSe₂, CdS, and ZnO.

Although a variety of crystalline inorganic semiconductor deposition methods exist, not all methods are suitable for use in depositing over a flexible substrate as the processing conditions associated with some deposition methods may result in damaging or destroying the flexible substrate. Advantageously, solution processing methods may allow for deposition of thin layers of crystalline inorganic semiconductor material over the flexible substrate under conditions that do not result in damage to the flexible substrate. In some embodiments, the crystalline inorganic semiconductor material is deposited over the substrate using a solution processing method, such as a spray coating method, a spin coating method, a drop casting method, a spin coating method, an inkjet printing method, or a doctor blading method.

For example, a suspension of crystalline nanoparticles may be deposited over the flexible substrate and solvent of the suspension may be allowed to evaporate. In this way, a layer of dried crystalline nanoparticles may be formed.

Use of a solution processing method for depositing the crystalline inorganic semiconductor material over the flexible substrate may also provide other advantages. For example, in some embodiments, the crystalline inorganic semiconductor material conformally or intimately contacts nano features of the flexible substrate, such as raised regions or recessed regions, and may be fabricated in such a configuration using a solution processing method. In some embodiments, the conformal or intimate contact is direct, and the crystalline inorganic semiconductor material is in direct contact with the flexible substrate. In some embodiments, the conformal or intimate contact is indirect, and one or more other materials are positioned between the crystalline inorganic semiconductor material and the flexible substrate, though adjacent layers may be in conformal or intimate contact with one another.

Crystalline inorganic semiconductor material may take on any suitable dimensions. In some embodiments, the crystalline inorganic semiconductor material has a thickness of about 25 nm to about 250 nm. In some embodiments, the crystalline inorganic semiconductor material has a lateral dimension of between about 10 nm and about 10 cm.

In some embodiments, the crystalline inorganic semiconductor material is flexible. Flexibility of the crystalline inorganic semiconductor material may be due to the thickness of the material. Flexibility of the crystalline inorganic semiconductor material may be due to the nanocrystalline structure, such as a nanoparticle or nanowire structure, where individual nanoparticles or nanowires can slip or move past one another, relieving stress or strain in the material and minimizing or reducing a likelihood of fractures, cracks, or other destructive features from forming.

Like the flexible substrate, in some embodiments, the crystalline inorganic semiconductor material may itself be flexible. For example, the crystalline inorganic semiconductor material may optionally undergo bending to a radius of curvature of 3 mm or more without sustaining damage associated with impaired device performance, such as a radius of curvature between 3 mm and 5 mm, between 3 mm and 6 mm, between 3 mm and 7 mm, between 3 mm and 8 mm, between 3 mm and 10 mm, between 3 mm and 15 mm, between 3 mm and 20 mm, between 3 mm and 25 mm, between 3 mm and 50 mm, or between 3 mm and 100 mm. In some embodiments, the crystalline inorganic semiconductor material is free of cracks or fractures that cause failure or short circuiting of the electronic device component, even while in a curved or bent configuration.

### VI. Example Electronic Devices

A variety of flexible electronic devices are provided. In some embodiments, the flexible electronic device comprises a circuit. For example, the electronic device components of the flexible electronic device may comprise circuit elements, such as resistors, capacitors, inductors, diodes, semiconductor junctions, etc. A variety of circuit elements may be constructed over the flexible substrate in any desirable configuration to create a useful electronic circuit.

Specific example electronic device components useful for various applications include, but are not limited to, a semiconductor junction, an inorganic light emitting diode, a thin film transistor, a memory device, an optical detector, a magnetic device, and a photovoltaic device.

The electronic device components may be assembled into a variety of different flexible devices useful for a variety of applications. Useful applications include, but are not limited to a label, a sticker, a poster, a textile, a sensor, a body-integrated device, a drone, a photovoltaic tape, a photovoltaic wallpaper, a photovoltaic surface applique, a photovoltaic window covering, a man made structure, such as a building or a portion thereof, and an electronic display.

In some embodiments, the flexible electronic device comprises a flexible photovoltaic device. For example, in some embodiments, the electronic device component may comprise a photovoltaic cell. The inventors have identified a set and ordering of materials for creating a flexible photovoltaic cell compatible with a cellulose nano-fiber based paper substrate.

For example, in some embodiments, the electronic device component comprises a bottom electrode supported by the flexible substrate, a first semiconductor material in electrical contact with the bottom electrode, a second semiconductor material in electrical contact with the first semiconductor material, and a top electrode in electrical contact with the second semiconductor material. In some embodiments, one of the first semiconductor material and the second semiconductor material comprises an n-type semiconductor, and one of the first semiconductor material and the second semiconductor material comprises a p-type semiconductor. In this way, the two semiconductors may form a p-n junction, which is useful for different applications, including creation of a photovoltaic device.

In some embodiments, the bottom electrode comprises a first conductor, which may optionally be a transparent conductor. In some embodiments, the top electrode comprises a second conductor, which may optionally be a transparent conductor. Optionally, one of the first semiconductor material and the second semiconductor material comprises CdS. Optionally, one of the first semiconductor material and the second semiconductor material comprises CuInSe₂. Optionally, at least one of the first conductor and the second conductor comprises a transparent conductor.

The inventors have demonstrated a large power conversion efficiency using the photovoltaic cells of some embodiments. It will be appreciated that a variety of different power conversion efficiencies are achievable using the photovoltaic cells. For example, in some embodiments, the photovoltaic cell exhibits a power conversion efficiency of about 1% or more, about 1.25% or more, about 1.5% or more, about 1.75% or more, about 2% or more, about 2.25% or more, between about 1% and about 2.25%, between about 1% and about 2.5%, between about 1% and about 3%, or between about 1% and about 3.5%. For some applications, a power conversion efficiency of less than or about 1% may be suitable, and the disclosed devices may provide for such efficiency levels.

### VII. Methods of Making Flexible Devices

The inventors have developed methods for making flexible devices, such as those described above. Initially, a flexible substrate is provided, such as a flexible substrate comprising paper including cellulose nano-fibers having average diameters between about 50 nm and about 150 nm. Next, one or more flexible device components may be deposited over the substrate. In some embodiments, the flexible device components may include at least a crystalline inorganic semiconductor material.

In some embodiments, providing the flexible substrate may include purchasing or obtaining the flexible substrate from an outside source. In some embodiments, providing the flexible substrate may include making the flexible substrate.

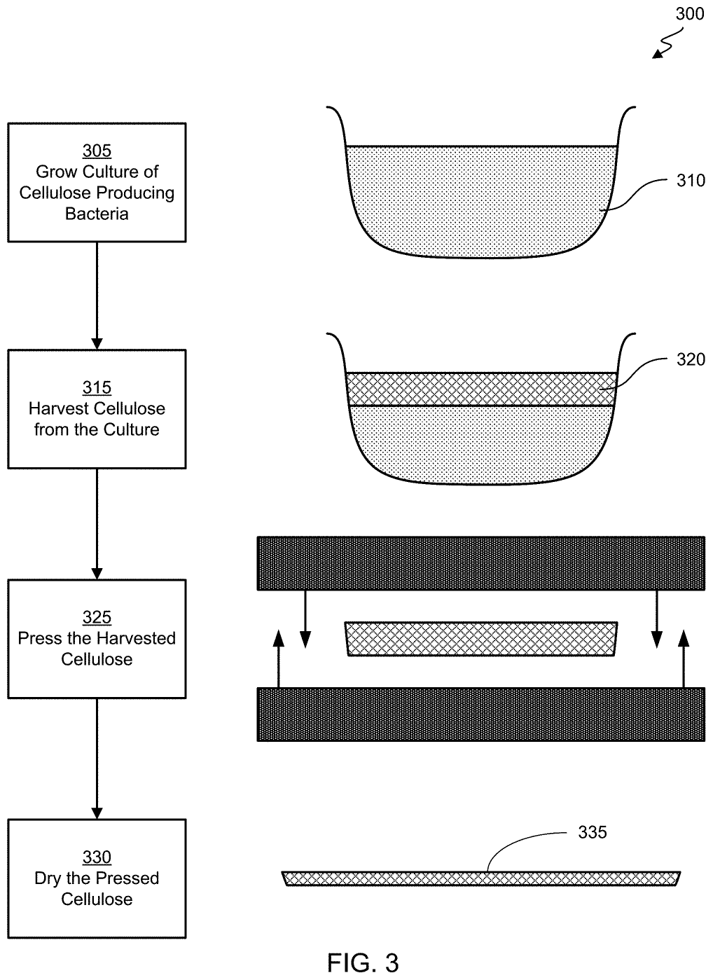

FIG. 3 provides a schematic overview of a method 300 for making a flexible substrate in accordance with some embodiments. Initially, at block 305, a culture 310 of cellulose producing bacteria is grown. As described above, a variety of cellulose producing bacteria are useful for making flexible substrates in accordance with some embodiments. Example bacteria include, Gluconacetobacter hansenii. It will be appreciated that suitable conditions may be required for growing the cellulose producing bacteria culture.

At block 315, the cellulose 320 from the culture is harvested. The cellulose may be in the form of a pellicle, or as an agglomeration of crystalline cellulose. In some embodiments, the cellulose may be washed, rinsed, or otherwise processed before proceeding further.

At block 325, the harvested cellulose 320 is pressed. It will be appreciated that pressing the harvested cellulose 320 may aid in formation of a web or membrane to transform the cellulose into paper. In some embodiments, pressing the harvested cellulose 320 may aid in the removal of water from the cellulose.

At block 330, the cellulose paper 335 is dried. In some embodiments, this step may be optional, as certain processing conditions do not require the cellulose paper 335 to be dried. A variety of drying conditions may be used. In some embodiments, a cold or hot air blower or drier may be employed. In some embodiments, the cellulose paper 335 may be exposed to the atmosphere to allow natural evaporation processes to dry the cellulose paper 335.

Once the flexible substrate is available, the method of making the flexible electronic device may continue. For example, as described above, one or more flexible device components may be deposited over the substrate, such as a flexible device component comprising crystalline inorganic semiconductor material.

As described above, a variety of processing conditions are useful for depositing a crystalline inorganic semiconductor material over the flexible substrate. In some embodiments, the crystalline inorganic semiconductor material is deposited over the flexible substrate using a solution processing method.

It will be appreciated that the one or more flexible device components may include other materials beyond crystalline inorganic semiconductor materials. For example, in some embodiments, the one or more flexible device components further comprise one or more conductors. For example, one or more conductors may be deposited over the flexible substrate using a solution processing method or a dry deposition method. Dry deposition methods useful with the methods of making flexible electronic devices may be compatible with the flexible substrate, in that they may not destroy or damage the flexible substrate.

Useful dry deposition methods include, but are not limited to a physical vapor deposition method, an evaporation deposition method, thermal evaporation, a sputtering deposition method, radio frequency sputtering, and any combination of these. It will be appreciated that different dry deposition methods may be employed depending on the chemical identity and compatibility of the material being deposited.

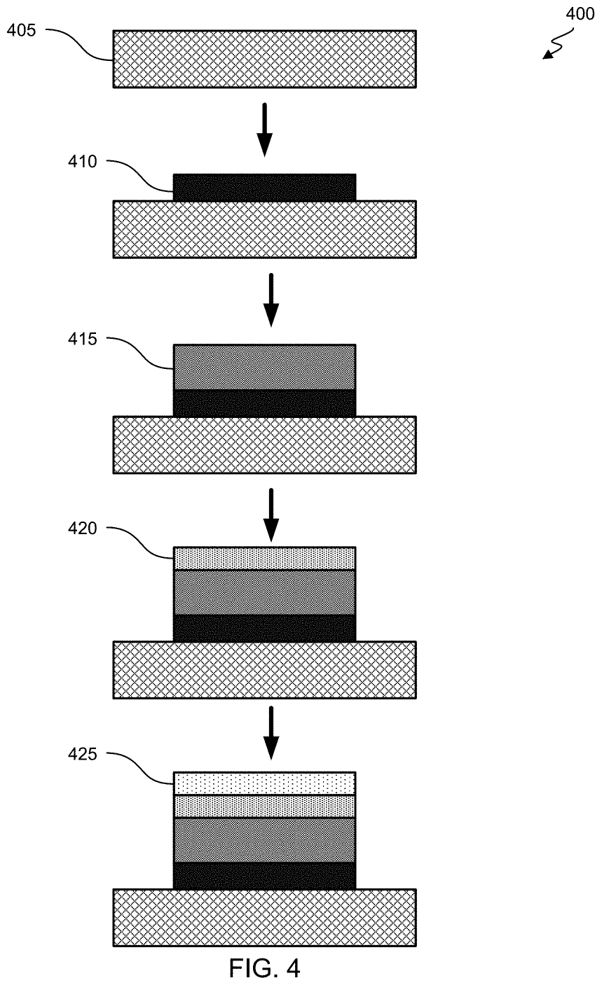

FIG. 4 provides a schematic overview of a method 400 of making a flexible electronic device in accordance with some embodiments. In FIG. 4, the flexible electronic device shown being made is a photovoltaic cell. Initially, a flexible substrate 405 is provided. As described above, the flexible substrate 405 may be made or obtained from an outside source.

Next, a bottom conductor 410 is deposited over the flexible substrate. In some embodiments, the bottom conductor 410 may comprise a multilayer or composite conductor. For example, in one embodiment, a first layer of chromium may be thermally evaporated onto the substrate and a second layer of gold may be thermally evaporated onto the chromium layer. In some embodiments, the bottom conductor 410 may comprise a transparent electrode, such as indium tin oxide, which may be deposited using a radio frequency sputtering method, for example.

Next, a first semiconductor layer 415 is deposited over the bottom conductor 410. For example, in one embodiment, first semiconductor layer 415 may comprise a layer of CuInSe₂ nanocrystals deposited using a solution processing method. For example, a spray coating method may include spraying a suspension of CuInSe₂ nanocrystals in toluene onto and allowing the toluene to evaporate from the sprayed material.

Next, a second semiconductor layer 420 is deposited over first semiconductor layer 415. For example, in one embodiment, second semiconductor layer 420 may comprise a layer of CdS deposited using a solution processing method. For example, a precursor solution may be dropped onto the first semiconductor layer 415 and then the precursor solution heated to drive a chemical reaction to form the second semiconductor layer 420.

Finally, a top conductor 425 is deposited over the flexible substrate. In some embodiments, the top conductor 425 may comprise a multilayer or composite conductor. For example, in one embodiment, the top conductor 425 may comprise a transparent conductor, such as ITO, which may be deposited using a radio frequency sputtering method, for example.

It will be appreciated that additional processing steps that are not recited here may be used for making flexible electronic devices in accordance with the present disclosure. For example, one or more additional deposition steps may be used. In addition, sacrificial materials may be utilized, such as for masking purposes, to enable desired shapes of deposited materials to be achieved. Further, each deposition process identified may correspond to one or more deposition sub-processes, such as to deposit an element that may comprise multiple layers. For example, in some embodiments, the second semiconductor layer 420 may comprise multiple semiconductor sub-layers.

### VIII. Methods of Using Flexible Devices

The flexible devices described above, including those made by the above described methods, are useful for a variety of applications. Example applications for the flexible devices include, but are not limited to, use in a label, a sticker, a poster, a textile, a sensor, a body-integrated device, a drone, a photovoltaic tape, a photovoltaic wallpaper, a photovoltaic applique, a photovoltaic window covering, a man-made structure or a component thereof, or an electronic display. In some embodiments, the flexible devices include a photovoltaic cell, which may provide power to other devices or be used to provide power to other components of the flexible devices.

In some embodiments, methods of operating a flexible electronic device may comprise providing current or voltage for use by the flexible electronic device component. Currents or voltages may be provided through one or more electronic contacts, for example, or may be provided by a battery cell or photovoltaic cell integrated into or attached to the flexible electronic device.

In some embodiments, methods of operating a flexible electronic device may comprise generating a current or voltage using the flexible electronic device component. For example, in embodiments where the flexible electronic device comprises a photovoltaic cell, the device may be exposed to light of a sufficient energy and intensity to generate a flow of electrons. These electrons may be directed to another electronic device or device component for use, such as to power a display, power a motor, power a circuit, power a processor, etc.

In some embodiments, methods of operating a flexible electronic device may comprise generating current or voltage using a first portion of the electronic device component for use by a second portion of the electronic device component. In this way, the flexible electronic device may be self-powering. That is, a power source, such as a photovoltaic cell, may be included in one portion of the flexible electronic device, and electric current or voltage generated by the power source may be used to provide power to another component of the flexible electronic device, such as a display, transmitter, processor, sensor, circuit, etc.

It will be appreciated that the flexible device may be bent, flexed, or stretched while in use and the device may continue operating. For example, the flexible substrate may be bent to a radius of curvature of 3 mm or more, between 3 mm and 5 mm, between 3 mm and 6 mm, between 3 mm and 7 mm, between 3 mm and 8 mm, between 3 mm and 10 mm, between 3 mm and 15 mm, between 3 mm and 20 mm, between 3 mm and 25 mm, between 3 mm and 50 mm, or between 3 mm and 100 mm. Optionally, the flexible substrate may be stretched in a lateral direction, such as to 100%-150% of an unstretched lateral size. Optionally, the flexible substrate may be folded and/or unfolded.

Other, more complex methods of operating flexible electronic devices may be performed within the scope of this disclosure. At a basic level, however, most or all of the methods of operating flexible electronic devices may encompass or make use of the aspects described above relating to receiving or providing electric power. For example, in some embodiments, the flexible electronic device component may generate a display, or obtain or provide a sensor reading. Such aspects may require use of electric current or voltage for actuating the display elements or sensor elements.

### IX. Examples

The invention may be further understood by the following non-limiting examples.

### Example 1: Flexible CuInSe₂ Nanocrystal Solar Cells on Paper

Solar cells on paper have great potential to be both inexpensive and portable, due to several unique features of the substrate; paper is cheap, flexible, lightweight, biodegradable, and manufactured by roll-to-roll processing. This example describes a nanocrystal (NC) photovoltaic (PV) device on a paper substrate with a power conversion efficiency (PCE) of 2.25%. The paper substrates in these devices comprise or are composed of bacterial cellulose nano-fibers, synthesized by the microorganism Gluconacetobacter hansenii. CuInSe₂ (CIS) nanocrystals are used as the absorber material, which are deposited by spray coating. These devices have demonstrated exceptional electrical and mechanical stability, showing no significant loss in performance even after more than 100 flexes to 5 mm radius. The devices even performed when folded into a crease. Moreover, practicality and advantages of these flexible paper photovoltaics (PVs) are demonstrated by building a prototype device that powers a liquid crystal display (LCD) screen while mounted to a variety of surface types. This example provides a processing paradigm for fabricating flexible CIS nanocrystal PVs on paper substrates.

Transistors, light emitting diodes, electrothermochromic displays, microfluidic devices, and touch pads may be fabricated on paper, providing the backdrop to creation of full electronic devices on paper. One difficulty encountered in this subfield of research has been the lack of a lightweight portable power supply. As all electronic devices require power, either hardwiring a power supply or adopting a heavy battery system counter-intuitively defeats the purpose of using a lightweight and portable substrate. The solar devices developed in this Example provide an ideal power solution for use by other electronic components on paper.

Beyond portable electronics, paper solar cells have many advantages over conventional PVs constructed on glass or on flexible substrates like metal foils and plastics. Paper substrates offer far better solutions given cost, consumer adoptability, eco-friendliness, biodegradability, and ease of recycling. Conventional lamination techniques can be readily adopted to encapsulate paper solar cells from damage as a result of handling and moisture. Beyond these factors, paper PVs are extremely lightweight, allowing for cheap and simple shipping and installation, unlike glass-mounted solar panels. Furthermore, roll-to-roll manufacturing of solar cells dramatically increases the throughput, reducing the cost, time, and complexity required for processing. From a manufacturing perspective, these paper PVs have the distinct advantage over conventional glass-mounted solar panels in that they can be cultivated and produced with little more than a simple wet laboratory set up, enabling for on-site fabrication. This creates an important advantage over conventional PVs made with glass substrates in circumstances where the complexity, cost of freight, and the weight of the panel becomes a limiting factor.

Thus far, the only paper solar cell technologies that have had any degree of success have been organic photovoltaics (OPVs) and dye-sensitized solar cells (DSSCs). OPVs and DSSCs have the benefit of being fabricated under mild conditions by solution processing, but still have problems with stability and long term performance. OPVs undergo photo-oxidative reactions, and are unstable under light exposure and in the presence of oxygen. DSSCs use a liquid electrolyte, which is temperature sensitive, and also not stable. Nanocrystal PVs, on the other hand, have advantages, such as low cost, mild processing conditions, reduced manufacturing complexity, and easy scalability, but also exhibit better thermal and air stability, as well as high carrier mobilities than OPVs or DSSCs. This Example describes a nanocrystal PV device fabricated on a paper substrate. The devices achieve an exceptional power conversion efficiency of 2.25%, higher than any other solar cell fabricated directly on paper. The paper substrate used in this Example is made from pure crystalline cellulose nano-fibers synthesized by the microorganism Gluconacetobacter hansenii and is referred henceforth in this Example as bacterial cellulose paper.

Additionally, bacterial cellulose has a variety of interesting material applications. It is useful as temporary skin in medical care, in acoustic diaphragms, as conductive carbon films, as a separation medium, as an electronic paper display, as a substrate for organic light emitting diodes and, as described here, as a substrate for solar cells, which provides a basis for formation of other active crystalline electronic device components on paper. Its use as a wound dresser demonstrates its compatibility with the human body, allowing solar cells on microbial cellulose to be adhered directly to the skin enabling incredible portability, ease of installation, and integration into everyday life. The proven compatibility of bacterial cellulose with the human body indicates a great potential for its use in the development of prosthetic, body-integrated electronic devices. The attributes of bacterial cellulose enables extreme integration of electronic devices, not only in the human body, but also in infrastructure, devices, and sensors related to the “internet of things,” and data-mining devices for dynamic field research practices.

Using CuInSe₂ (CIS) nanocrystals as an absorber material, the devices demonstrated a very high degree of flexibility and did not show any degradation in device performance after more than 100 tensile bending cycles to a radius as small as 5 mm. Another type of solar cell, flexible CIGS solar cells, has focused on flexible substrates with target performance levels similar to those obtained on glass, but has not focused on substrates that can actually allow solar cells to be flexible without causing degradation in performance. CIGS solar cells of this nature may be bent to a bending radius only as small as 2 cm without considerable performance loss. The nanocrystalline PV cells described here demonstrated a much smaller bending radius of 3 mm without any loss in device performance. It should be noted that achieving this high flexibility has been performed without substituting the brittle ITO with a flexible transparent conducting electrode. The high flexibility achieved may be attributed to the use of bacterial cellulose as the substrate and CIS nanocrystals as the absorber material.

Be it convex, concave or complex, flexible cellulose paper PVs can be attached to any surface curvature. This degree of flexibility allows for paper-based solar panels to be mounted at sites where conventional flat heavy panels are difficult or expensive to install, both reducing the cost of installation and dramatically increasing the ability for solar technology to be intelligently integrated into architecture and industrial design. In architectural application these PVs can be directly adhered onto walls, enhancing aesthetic building solutions, and increasing design options in urban planning. Given that the CIS nanocrystal layer can be printed, the flexibility of these devices allows for paper photovoltaics to function as custom designed PV wallpaper for both interior and exterior applications.

Furthermore, the solar devices described in this Example have the unique property of functioning even when folded and creased. In the experiments described, these paper-based devices functioned not only when the substrate was folded adjacent to the active areas of the solar cells, but also surprisingly even when folded across the surface of the device itself. This degree of foldability and flexibility provides a platform upon which to develop a new class of portable electronics and photovoltaics that have the mechanical hardiness to be crinkled, creased, folded and adopted into ubiquitous use.

There are very few materials like paper that have the ability to maintain a crease when folded and hold its structure, enabling paper to improvise multiple structural configurations, as demonstrated by the practice of origami. Origami may be incorporated into photovoltaic design to maximize efficient absorption of sunlight. Previous devices in incorporating this design consideration, however, were not built on paper. With the use of paper devices, more complex origami structures may be easily exploited. While conventional pulp-processed paper has many beneficial features, the inventors have observed it to be an unsuitable substrate for nanocrystal PVs due to issues of high surface roughness and wettability issues with the solvents required for solar cell fabrication. Contrastingly, paper made from bacteria-cultivated nano-cellulose, or a man-made equivalent, can be engineered to avoid the aforementioned problems, enabling it to serve as an ideal substrate to make inexpensive, light-weight, and flexible solar cells.

Experimental Methods. Chemicals. Copper(I) chloride (CuCl, 99.99%), elemental selenium (Se, 99.99%), cadmium sulfate (CdSO₄, 99.99%), thiourea (99%), oleylamine (OLA; >70%), tributylphosphine (TBP; 97%), diphenylphosphine (DPP; 98%), carboxymethylcellulose, and anhydrous toluene (99.8%) were obtained from Aldrich; indium (III) chloride (InCl₃, 99.99%) was obtained from Strem Chemical; toluene, ethanol, hexanes, and ammonium hydroxide (18 M NH₄OH) were obtained from Fisher Scientific. 200 ml of a stock solution of oleylamine was degassed under vacuum overnight at 110° C. in a 250 ml glass 3 neck flask and stored in a nitrogen filled glovebox until further use. All the other chemicals were used as received.

CuInSe₂ nanocrystal synthesis. In the synthesis of CIS nanocrystals, 5 mmol of CuCl, 5 mmol of InCl₃, 1.5 ml of DPP and 50 ml of oleylamine were added in a 125 ml three-neck flask inside a nitrogen filled glovebox, which was subsequently sealed with rubber septa and brought out of the glovebox. The flask was then attached to a standard Schleck line and degassed at 110° C. for 30 minutes. The flask was then filled with nitrogen and the temperature maintained at 110° C. for 10 minutes. In the meantime, the selenium precursor solution was prepared by mixing 10 mmol of elemental Se with 10 ml TBP in a 20 ml glass vial equipped with a stir bar in a N₂ filled glovebox. This solution was let to stir for 20-25 minutes during which Se dissolved in TBP to give a clear solution. The Cu/In/DPP/oleylamine reactant solution was then heated to 180° C. while maintaining N₂ atmosphere, during which time the Se precursor solution was injected as quickly as possible using a 12 ml plastic syringe. The temperature was then raised to 240° C. quickly and maintained for 10 minutes. The heating mantle was then removed and the reaction mixture allowed to cool to room temperature. 20 ml of ethanol was added to this reaction mixture and centrifuged at 4500 for 10 minutes, discarding the yellow colored supernatant. Then a minimal amount of toluene was added to disperse the nanocrystals in the solid precipitate. 6 ml of ethanol was added to this dispersion and centrifuged at 4500 rpm for 10 minutes. The supernatant was discarded and the solid precipitate redispersed in a minimal amount of toluene and transferred to a 20 ml glass vial. Then this dispersion was rotovaped at 40° C. until all the toluene evaporated. The dried nanocrystals were moved into a N₂ filled glovebox, dispersed in anhydrous toluene to achieve a concentration of 100 mg/ml, and stored for further use.

Synthesis and preparation of cellulose substrates. The organism used for the present study was Gluconacetobacter hansenii ATCC 53582 strain NQ5 which produces a pure crystalline form of cellulose. Liquid cultures of G. hansenii NQ5 were grown to log phase until reaching an optical density of 2 at 600 nm. 10 ml of the resulting inoculum was added to a culture tray containing 500 ml SH media supplemented with 0%, 2%, or 4% carboxymethylcellulose (CMC). The trays were allowed to incubate under static conditions for 7 to 14 days at 28° C. The pellicles were harvested and the cells were removed using a 2% solution of Alconox (Sigma-Aldrich 242985) and stored in an aqueous solution of 20% Ethanol until further usage.

To prepare the membrane as a PV substrate, the pellicle was placed face down on a smooth Teflon sheet. The pellicle was compressed from the center outwards, in a radial manner to eliminate bubbles. A cotton fiber fabric was then placed on top of the cellulose and the aforementioned compression process was repeated to release any excess air between the materials. A sheet of Teflon was then placed inside a hydraulic press. A 15-ton hydraulic press was lowered gently and slowly onto the pellicle to eliminate over half of its water content, allowing for easier material handling and minimizing slippage. The plate and the fabric were then removed from the press leaving behind the compressed cellulose, face down on the Teflon sheet. A rectilinear stretching armature (made from surfaced pine wood) was placed face down and centered with the microbial cellulose. Each side of the cellulose was gently lifted and a staple placed into the wood armature starting from the center, working outwardly toward the corners. After each staple placement, more pressure was applied before placing the next, allowing for even surface tension on the cellulose, and uniform pressure on the armature. The corners were evenly folded, in a similar manner to the conventional method of mounting linen onto canvas stretchers in preparation for an oil painting. After the cellulose was stretched, it was dried with a hair dryer on low heat for approximately an hour, until the entire surface was dry. The substrate was left overnight at room temperature to dry completely.

PV Device fabrication and testing. The devices have a Au/CIS/CdS/ZnO/ITO architecture, fabricated on microbial cellulose substrates. Microscope glass slides were cut into 1″×1″ sizes and used as a support media for building solar cells on cellulose. The microbial cellulose substrates were mounted onto these glass slides by using either Kapton tape or PDMS. PDMS is prepared by heating a mixture of siloxane monomer and a cross linking agent (Sylgard Elastomer 184, Dow Corning Corporation) in 1:30 ratio and is applied to the supporting glass slide to 150° C. for 15 minutes. A 10 nm chrome layer and 80 nm gold layer were thermally evaporated onto these substrates followed by spray deposition of CIS nanocrystals diluted to 10 mg/ml with toluene as the solvent onto the substrates heated to 100° C. The spraying is done on the bacterial cellulose substrate with Sonotek ExactaCoat ultrasonic automated spray system, equipped with a 120 kHz ultrasonic nozzle, by rastering the spray nozzle across a rectangular area with 3 mm raster spacing, speed of 10 mm/sec, an ink injection rate of 0.1 ml/min, an air pressure of 1.6 psi, and the distance from nozzle to substrate set at 11.5 cm. A CdS layer was deposited onto the CIS nanocrystal layer by dropping 0.7 ml of CdS precursor solution (1.25 mL of 15 mM CdSO₄, 2.2 mL of 1.5 M thiourea, and 2.8 mL of 18 M NH₄OH in water) onto each of the 1″×1″ CIS deposited cellulose-glass substrates heated to 90° C. on a hot plate and covered with an inverted crystallization dish for 2 min. The substrates were then rinsed off with DI water and dried with compressed nitrogen. Top layers, i-ZnO and ITO were deposited by Radio Frequency (RF) sputtering from a ZnO target (Lesker, 99.9%) and a ITO target (Lesker, 99.99% In₂O₃:SnO₂ 90:10) respectively in Ar atmosphere. ZnO and ITO were deposited selectively onto 8 rectangular regions with active device areas varying from 0.08 cm² to 0.15 cm². Silver paint was then applied to have better electrical contact while testing.

The current-voltage characteristics were measured using a Keithley 2400 general purpose source meter and a xenon lamp source meter equipped with A.M. 1.5 filter. EQE measurements were made using a home-built spectrophotometer with lock-in amplifier (Stanford Research Systems, model SR830) and monochromator (Newport Cornerstone 260 ¼M), and calibrated with Si and Ge photodiodes (Hamamatsu).

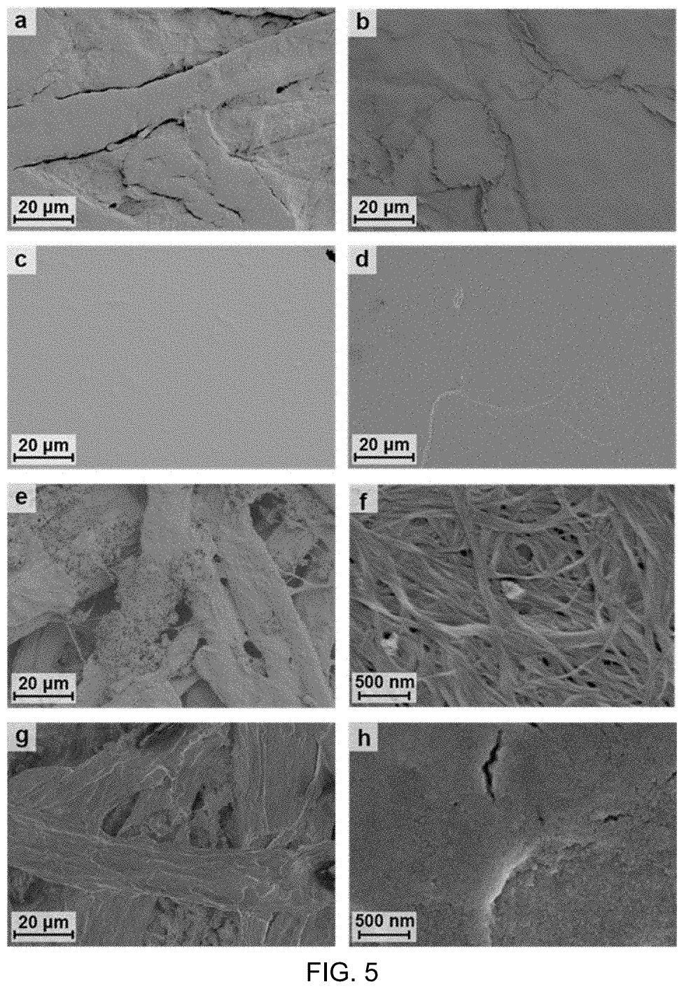

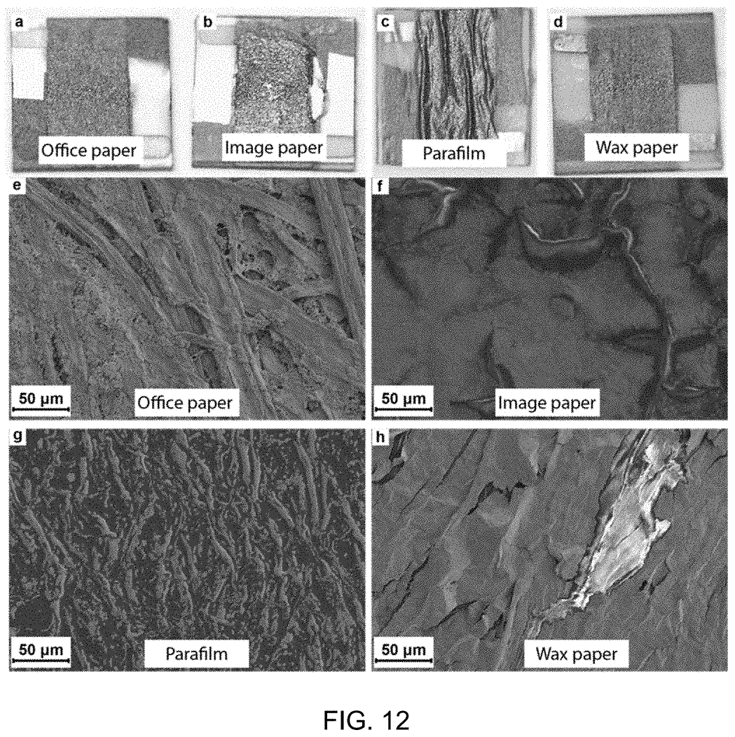

Results and discussion. Exploring various kinds of paper for device fabrication. Nanocrystal PVs were made on different kinds of paper. The surface topography of the paper was the determining factor in whether or not the devices were successful. FIG. 5 shows scanning electron microscope (SEM) images of different kinds of paper. In standard office paper and wax paper, the cellulose fibers in the material are quite coarse, generally more than 10 μm in diameter, as shown in FIG. 5, panel a and panel e. In parafilm samples, the cellulose fibers are not visible in SEM imaging because of plastic fillers, but other rough features can be seen in FIG. 5, panel b. The roughness on the micro scale of the substrate's surface topography for CIS nanocrystal PVs has proven problematic for functionality. Devices fabricated on these three kinds of paper have been observed to consistently short. While photo-printing paper is very smooth and has no rough features visible at the micro scale, it has poor adherence. The layers of the devices made on photo paper washed away while rinsing the substrates with DI water during CdS deposition step (see FIG. 12). In general, office, wax, parafilm, and photo-printing paper absorbed water and wrinkled during the CdS deposition step; SEM images and pictures of these four papers after CdS deposition are shown in FIG. 12. In order to avoid these problems with wetting, organic solar cells may be fabricated with all-dry deposition techniques. Bacterial cellulose, as described here, is a beneficial paper substrate for fabricating CIS nanocrystal solar cells, unique in its capacity to withstand all the wet processing steps while maintaining good adherence and exhibiting suitable surface roughness. Bacterial nano-cellulose fibers are much smaller in diameter (≈100 nm) than the cellulose fibers found naturally in plant material. Hence the substrate looks smooth at the micro scale (FIG. 5, panel d) while a rough network of cellulose fibers can be seen at the nanoscale (FIG. 5, panel f).

FIG. 5 shows the surface morphology of bacterial cellulose and office paper substrates coated with 80 nm gold, before and after the CIS layer deposition. In contrast to the cellulose fibers in office paper, the bacterial cellulose fibers are no longer visible after the CIS layer is deposited. Visibility of the fibers after CIS layer deposition implies that there is a high probability of having exposed back contact on the sides of the cellulose fibers and consequently the subsequent top layers come in contact with the bottom electrode layer causing shorts. This explains the reason for shorting of devices on office paper and functionality of devices on bacterial nano-cellulose paper substrates.

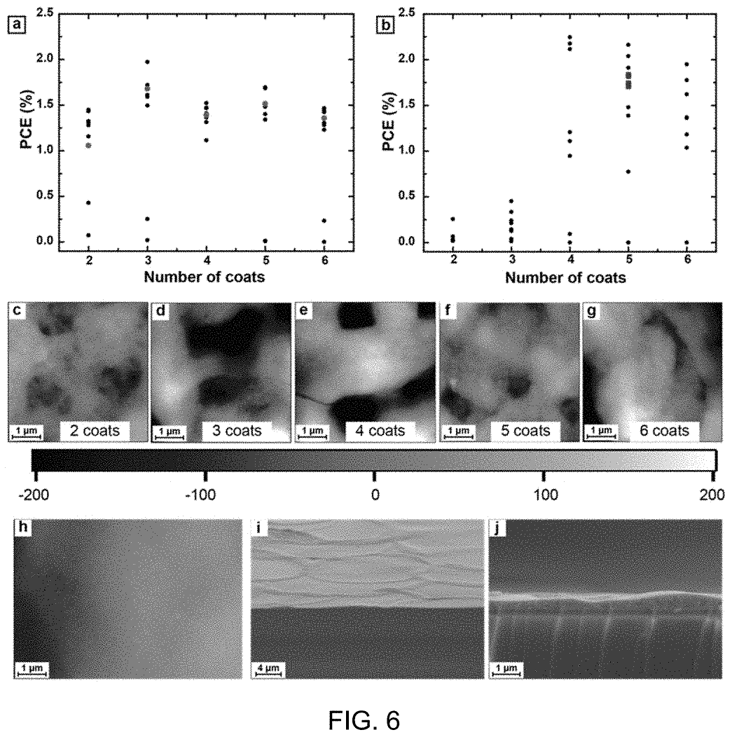

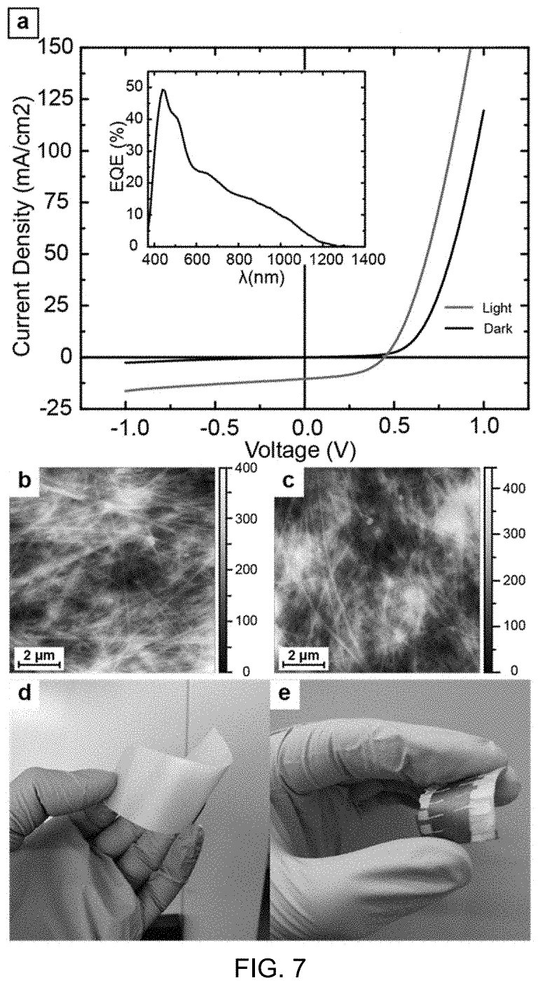

Influence of CIS layer thickness on device performance. FIG. 6 shows the plot of variation of PCE with the number of coats of CuInSe₂ nanocrystals sprayed using an automatic sprayer (spray parameters listed in the methods section). Number of coats is chosen as the parameter rather than the thickness because the uneven surface of the bacterial cellulose gives rise to a very uneven CIS layer and a single thickness value cannot be assigned to the CIS layer. Spraying with equivalent parameters on glass substrates deposits 50 to 100 nm of CIS layer with each coat. The uneven thickness of the CIS layer is caused by drying rings that form when the solvent (toluene) evaporates. CIS nanocrystal devices on glass when baked at 200° C. under vacuum for 10 minutes, show an improvement in PCE. A considerable amount of standard deviation is noted in the device performance both before and after baking as seen in the plots in FIG. 7. This is due to the uneven surface morphology of the CIS layer on bacterial cellulose, which can be seen in the Atomic Force Microscopy (AFM) images in FIG. 6 after 2, 3, 4, 5, and 6 coats of CIS nanocrystals. As mentioned above, it is evident from the AFM images as well, that the features of bacterial cellulose fibers are no longer present once coated with CIS nanocrystals. Uneven CIS layer simply means that different positions have different thickness of CIS, which directly impacts the power conversion efficiency. The CIS layer did not become uniform as the number of coats were increased and a considerable spread of PCE was observed even with increased number of coats where standard deviations for 4, 5, and 6 coats for devices after post baking are 37% (over 7 devices), 32% (over 6 devices), and 22% (over 6 devices), respectively, excluding the shorted devices. Even in devices fabricated on glass, the spray-coated CIS layer is not uniform in thickness because of the drying rings that form while the solvent toluene evaporates as shown in the SEM images in FIG. 6, panel i and panel j. Nanocrystal CIS PV devices on glass however, have a standard deviation of only 6.5% (over 4 devices) in PCE, considerably lower in comparison to comparable devices on bacterial cellulose, given the low variation in thickness and surface uniformity of glass. When compared to CIS on gold-coated bacterial cellulose at a similar scale, the AFM image of CIS nanocrystals on gold coated glass (FIG. 6, panel h) shows a much smoother surface morphology.

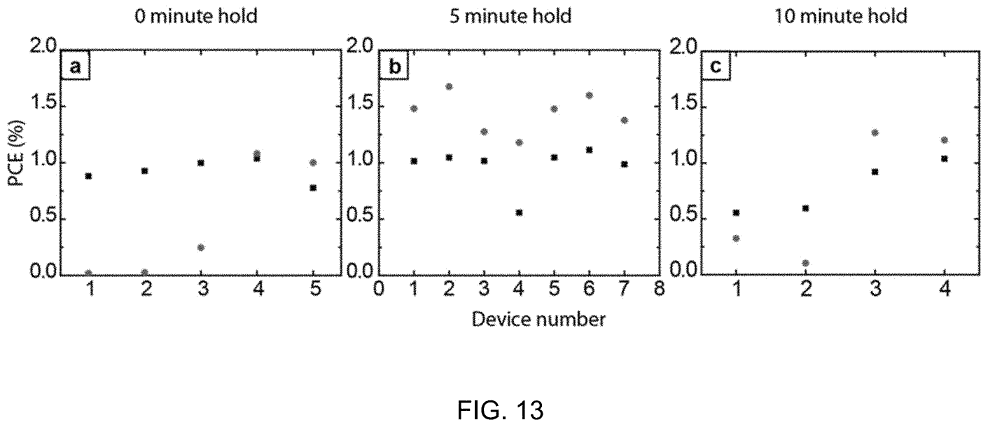

It can be inferred from the plot that the average PCEs of the non-baked devices decreases after 3 coats. This is because of the very thin active region (≈50 nm) in CIS nanocrystal devices, implying that even if more charge carriers are generated from thicker CIS layers, gains may be limited. The devices on cellulose were able to withstand the baking temperature of 200° C. The devices with thicker CIS layer performed much better after baking compared to devices with thinner CIS. This is again because of the rough surface of bacterial cellulose compared to other widely used smooth substrates such as glass. The presence of pinholes in the layers has a predominant effect in creating short circuits with baking when the CIS layer is thin. For these reasons the optimal baking time for glass may not be as useful for the paper substrates. The optimal baking time for the paper PVs investigated here was determined to be about 5 minutes; the details of the optimization experiment are provided below and in FIG. 13.

The inventors believe that the PCE of 2.25% reported here represents a significant improvement over any other paper solar cells. The previous best reported efficiency on paper was 1.3% with organic photovoltaic technology. 4% PCE on paper may be achievable by not directly fabricated on paper, but rather, fabricating on a zinc-coated polypropylene foil (functioning as back electrode) and then gluing that structure onto paper. FIG. 7, panel a shows the IV curve of a particular CIS nanocrystal device on bacterial cellulose. The roughness of these bacterial cellulose substrates varies from 50 nm to 100 nm. Atomic force microscopy images of this paper before and after gold coating are shown in FIG. 7, panel b and panel c. There is no change in the surface roughness after the deposition of gold layer. This surface roughness is one of the limiting factors for the PCE in these devices, considering that devices fabricated on glass achieve peak PCE at 3.1%. One device shown in this Example has a short circuit current (J_{sc}) of 10.47 mA/cm², an open circuit voltage (V_{oc}) of 0.44 V, and a fill factor (FF) of 0.48. A plot of external quantum efficiency (EQE) for this device is shown in the inset of FIG. 7, panel a. Short circuit current calculated from EQE measured under white light bias is 9.2 mA/cm², which is 12% less than the current measured from the IV curve and within reasonable limits. The power to weight ratio of these devices is around 0.35 to 0.4 Watts per gram, which is well within the range of the highest efficiency Si and triple junction solar cells constructed on glass, with the advantage of being much lighter in weight. Current Si and triple junction solar cells have a power to weight ratio of 0.82 and 0.39 Watts per gram.

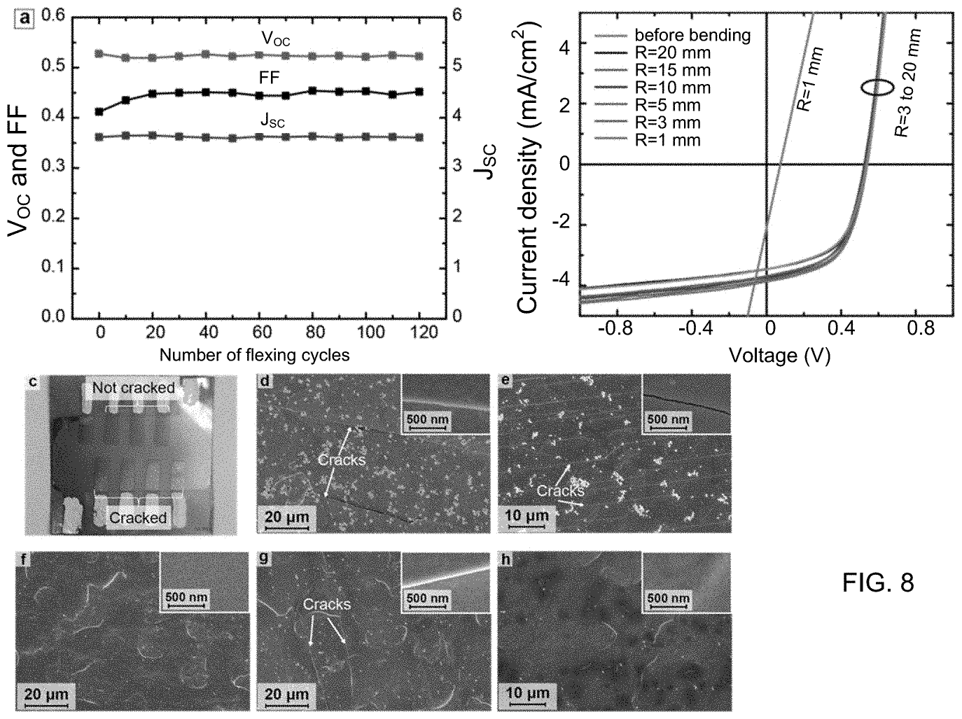

Flexibility of devices on cellulose. Research on making solution processed flexible solar cells has been primarily focused on organic photovoltaics. Traditionally, OPVs are fabricated by using ITO as the transparent conducting electrode, but the brittleness of ITO prevents the flexibility of the device and ‘flexible’ alternatives to ITO has been a focus of PV research. Carbon nanotubes, graphene, PEDOT:PSS, metal based transparent electrodes (Ag, Cu nanowires) may be used to replace ITO in OPVs, but the devices made with ITO as the transparent electrode may still exhibit a higher PCE than the alternatives, considering P3HT:PCBM organic PV systems. The CIS nanocrystal devices fabricated on PET with ITO as the top contact were shorted when bent to a radius of 10 to 15 mm, due to fractures in the ITO film and the CIS nanocrystal film, which can be seen in FIG. 8, panel c, panel d, and panel e. These cracks are large enough to be visible to the eye. FIG. 8, panel a is a plot of device characteristics of solar cells on bacterial cellulose as a function of number of flex cycles. The device characteristics are measured with the solar cells in flat configuration, after every ten flex cycles. Unlike the solar cells on PET, these solar cells demonstrated excellent electrical stability to mechanical deformations and did not show any visible ITO cracks even after bending to a radius of 5 mm for more than 100 times, as can be seen in the SEM image in FIG. 8, panel f. The curved line features seen in the SEM images of FIG. 8, panel f, panel g, and panel h are due to the uneven thickness of the ITO that is caused by the rough bacterial cellulose substrate and the uneven CIS layer, and are not cracks. There was no significant change in performance, even after 120 tensile flexing cycles at 5 mm bending radius. This feature makes bacterial cellulose a useful substrate to fabricate solution processed flexible PVs without sacrificing the use of ITO as the top electrode. FIG. 8, panel b is a plot of IV curves of device on bacterial cellulose after being flexed to different radii and tested after returning it to flat position. It can be seen that the device is stable after being bent to a radius as low as 3 mm. Bending it even further to a radius of 1 mm destroyed the device. On examining the failure mechanism of the devices constructed on bacterial cellulose subjected to a bending of 1 mm radius, cracks were observed in the ITO layer as shown in the SEM image in FIG. 8, panel g. This cracking of the ITO layer led to a decrease in the solar cell performance. But unlike the devices on PET, these devices showed no evidence of large cracks that propagate through the CIS layer (FIG. 8, panel h). A few localized cracks that are visible were present right from the moment the CIS layer is deposited and are a result of the rough bacterial cellulose surface. This ability of the nanocrystal CIS layer on bacterial cellulose to refrain from cracking left only the brittle ITO layer with the challenge, and the ITO layer here is able to withstand much more bending stress since there are no large cracks in the CIS layer to initiate early cracks in the ITO layer. This is one difference that has given the ability for CIS NC PV devices on bacterial cellulose to withstand extreme tensile bending stress. Without wishing to be bound by any theory, the inventors believe that the ability to withstand cracking may be attributable to the strong adherence between the nanocrystals and the cellulose substrate caused due to trapping of the nanocrystals in the nano features of the bacterial cellulose substrate.

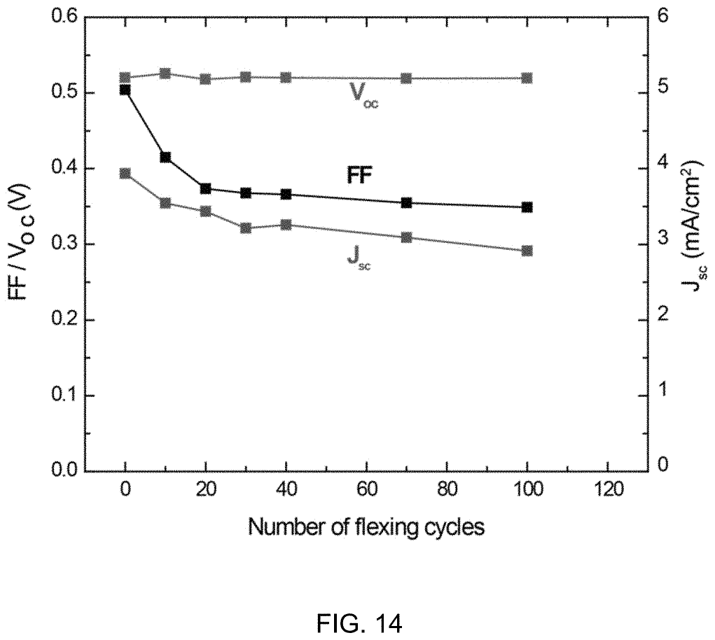

One problem faced while fabricating these devices was the difficulty of unmounting the completed devices from glass. Throughout device fabrication, the bacterial cellulose substrate was adhered to a glass slide for uniform processing temperatures. When Kapton tape was used as an adherent, it was not possible to unmount the devices after fabrication without destroying the device. It was important to identify an adherent that is stable at the processing temperatures and thermally conductive, but not so strong as to make the task of unmounting destructive. PDMS has all these required properties and is a useful glue for unmounting completed devices from glass. A loss in the device performance has been observed on repeated flexes to a radii lower than 5 mm and the trend is shown in FIG. 14.

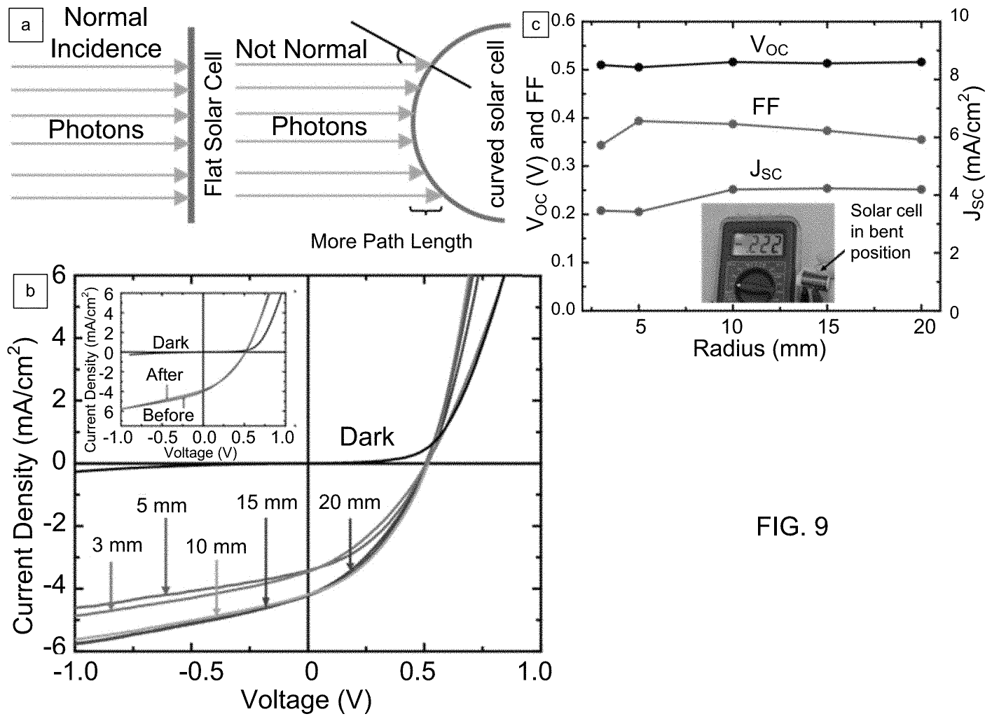

The performance of devices while under stress (rather than while flat) has also been examined. FIG. 9, panel b shows the IV curves of a device taken while it is being bent to various radii. There are various reasons that can affect the IV curves while carrying out such a measurement. Notably, the path traveled by the light may be different at different points of the solar cell and the incident light is no longer normal throughout the area of the device. Hence the device parameters, especially the short circuit current, which is highly affected by the intensity of light falling on the solar cell, may be influenced by such measurements even though there is no mechanical deformation in the layers of the solar cell. Due to the aforementioned reasons, whose effect becomes more and more prominent with the decrease in the bending radius, the short circuit current is recorded to be lower than the starting value for bending radius of 5 mm and 3 mm as can be seen in the plot in FIG. 9, panel b. The IV characteristics of this device in flat orientation before and after being subjected to the bending study are measured to be similar as can be seen in the inset in FIG. 9, panel b, proving the electrical stability to deformation at various bending angles.

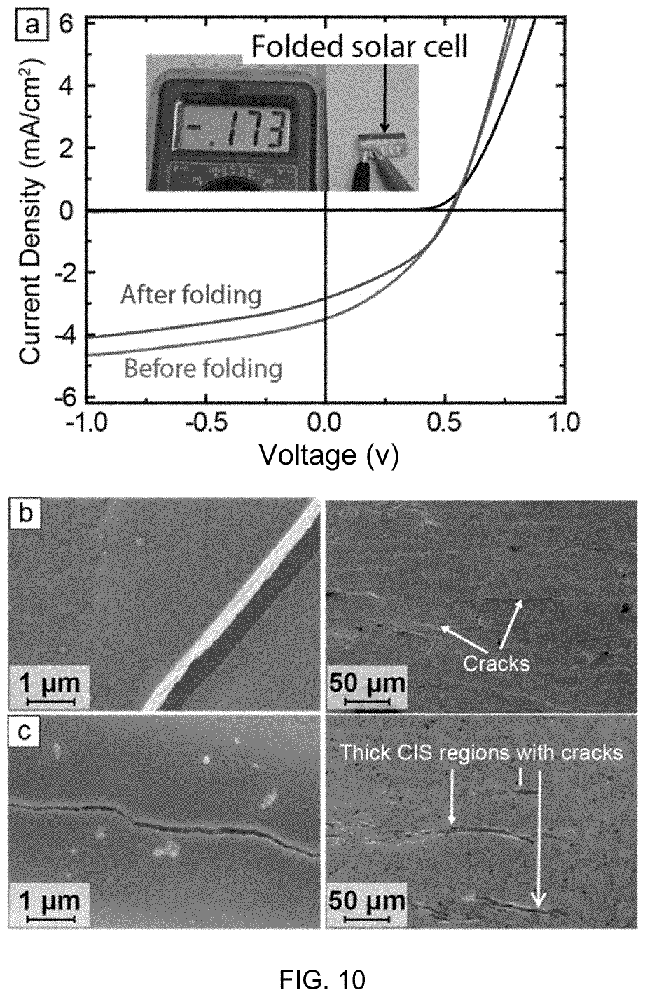

These solar cells were further tested for foldability. Four devices have been folded and the JV curves have been measured, with one of the four devices able to retain 86% of the efficiency even after folding and unfolding. Until now, the research on foldable solar cells has been focused primarily on folding in places where there are no device layers. In these devices, a solar cell has been folded and made to hold a crease through the architecture of the device layers, and has maintained functionality. This device showed PV response for 5 cycles of folding and unfolding. SEM analysis has been done on CIS nanocrystal layer and the ITO layer to understand the failure modes. As expected, cracking has been observed in the ITO layer (FIG. 10, panel b), but only a few cracks are observed in the CIS nanocrystal layer (FIG. 10, panel c). The cracking in the nanocrystal layer is observed only in areas where there is a thick CIS layer (lower magnification SEM image in FIG. 10, panel c), while all other areas remained crack-free. The voltage of this device has been measured while being folded in room lighting using a voltmeter and it has been observed that its voltage decreases considerably while its folded, but retains all the voltage after unfolding. The fall in performance is due to discontinuity caused in the ITO film while folded, which becomes continuous after unfolding. All the other layers remained almost intact upon folding except the ITO layer. Replacing the brittle ITO layer with flexible transparent conducting electrodes like PEDOT:PSS, graphene, nanowires, etc., may permit the device to function even while folded. Such foldability may provide advantages for portability of solar cells. This feature may allow paper solar cells to be folded for transportation and spread open when power extraction is desired.

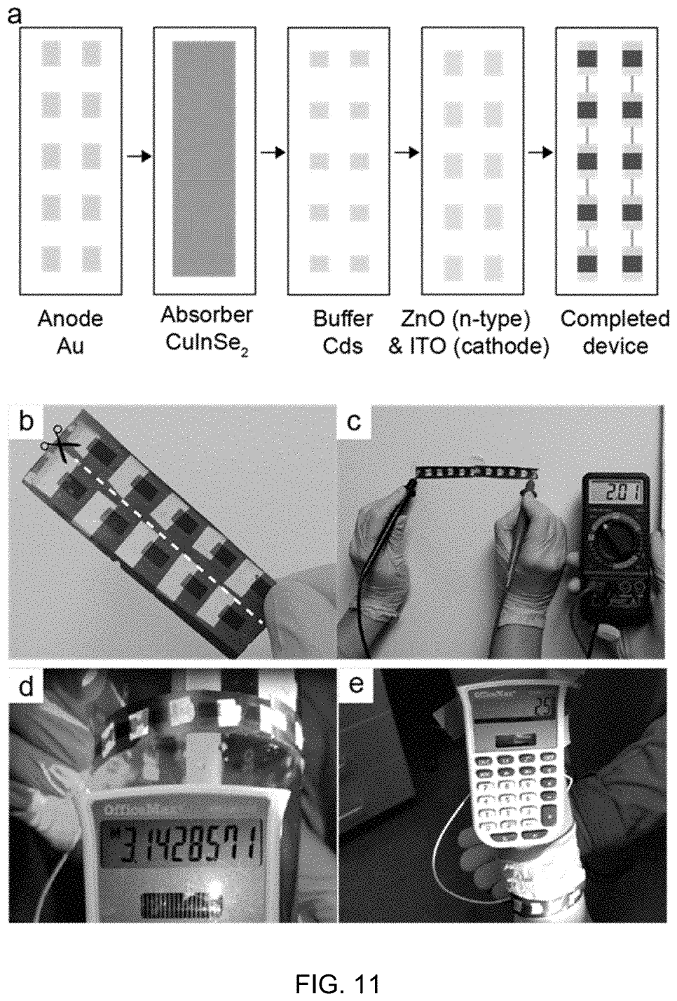

Solution processing allows “printing” of patterned device layers on any substrate, which enables easy integration of a number of cells in series or parallel. To demonstrate the simplicity of the process, strips of CIS devices on cellulose have been fabricated to power electronic devices. FIG. 11, panel a shows the patterning of the different layers on a strip of CIS device fabricated on cellulose substrate. Ten devices with areas of 0.1 cm² were fabricated on a 3″×1″ bacterial cellulose substrate, with the ability to be wired in either series or parallel configurations. To demonstrate the ease of installing these devices, the devices were cut as shown in FIG. 11, panel b, breaking it midway into two strips of 5 cells each and connecting all 10 devices in series. This device was mounted to both flat and curved surfaces to power electronic devices as shown in FIG. 11 panels d, e, and f Each row delivers 1.5 V in average indoor fluorescent lighting when connected in series, which is near summation of voltages from all the individual small devices. A relatively small device area was chosen as it allows for easy buildup of voltage (by connecting cells in series) while also reducing the possibility of shorting. Both the flexibility and the ability to power electronics have been demonstrated by sticking the flexible CIS cellulose devices onto a variety of surfaces including the complex curvature of a water bottle and around a human wrist. In multiple surface mountings, a calculator was powered and tested to give accurate summations.

Conclusions. Different types of paper substrates have been investigated for use as a substrate in nanocrystal PVs. Nanocrystal PVs were fabricated on a paper substrate. Moreover, the power conversion efficiency of the CIS nanocrystal PV on bacterial cellulose reported in this Example is among the highest for paper solar cells. With the combination of bacterial cellulose as the substrate and CIS nanocrystals as the absorber layer extremely flexible CIS/CIGS solar cells were fabricated, supporting the fabrication of these devices in a roll-to-roll process, allowing reduced PV fabrication complexity and decreased production costs.

Furthermore, the fabrication of microbial cellulose is a process that can be semi-portable and enable on-site fabrication of PV cells where the resources of a wet laboratory are available. The process through which these solar cells are fabricated lends itself to development in extremely off-grid sites, such as where the cost of shipment and consequently the weight of the solar cells becomes a limiting factor. Thus, solar cells made on a bacterial cellulose paper substrate provide a novel solution to the need for low complexity, low cost, low power supplying PVs, and have the added benefits of being flexible, readily integrated for prosthetic use with the human body, and capable of on-site fabrication.

Figure Captions. FIG. 5. SEM of various kinds of paper (panel a) wax paper (panel b) parafilm (panel c) photo printing paper (panel d) bacterial cellulose (panel e) office paper (panel f) zoomed in image of bacterial cellulose. (panel g) CIS coated office paper (panel h) CIS coated bacterial cellulose paper.

FIG. 6. Plots showing the effect of CIS layer thickness on device performance (panel a) before baking (data plotted in red is the average PCE, excluding the shorted devices) (panel b) after baking the completed devices at 200° C. for 5 minutes under vacuum in a rapid thermal processing (RTP) furnace (data plotted in blue is PCE of devices fabricated on glass), data from eight devices for each coat is plotted in black; panel c-panel g: AFM images with increasing coats of sprayed CIS layer on gold coated bacterial cellulose; panel h: AFM image of sprayed CIS layer on gold coated glass; panel i and panel j: SEM images of sprayed CIS layer on gold coated glass.

FIG. 7. Panel a: JV curve of one CIS NC PV device on bacterial cellulose, which has a PCE of 2.25%, J_{sc} of 10.47 mA/cm², V_{oc} of 0.44 V, and FF of 0.48; panel b and panel c: AFM images of bacterial cellulose substrates before and after depositing 80 nm of gold; panel d: photograph of bacterial cellulose paper; panel e: a lab scale CIS NC PV device on bacterial cellulose with 8, 0.1 cm² devices on a single substrate.

FIG. 8. Panel a: plot of average device characteristics as a function of flex cycles (R=5 mm) for four CIS NC PVs on bacterial cellulose; panel b: JV curves of CIS NC PV device on bacterial cellulose in flat orientation after bending to various radii; panel c: CIS NC PV devices on PET, where the top devices have not been bent and the bottom side devices have been bent to 10 mm radii; panel d and panel e: SEM images of ITO and CIS NC layer of the device on PET after being bent to 10 mm radius; panel f: SEM image of ITO in the PV device fabricated on bacterial cellulose which has been flexed 120 times (R=5 mm); panel g and panel h: SEM images of ITO and CIS NC layer of the device on bacterial cellulose after being bent to 1 mm radius.

FIG. 9. Panel a: illustration showing the difference in the measurement in flat configuration Vs curved configuration; panel b: JV curves of CIS NC PVs on bacterial cellulose while being bent to different radii; panel c: device characteristics of CIS NC PV devices on bacterial cellulose measured while being bent plotted as a function of bending radii.

FIG. 10. Panel a: JV curve for CIS NC device on bacterial cellulose before and after folding, the inset shows is the same solar cell whose voltage is being measured in ambient room lighting with a voltmeter; panel b: SEM images of ITO layer in the CIS NC PV device on bacterial cellulose that is folded showing the cracks; panel c: SEM image of CIS NC PV device on bacterial cellulose showing the thick CIS regions with cracks.

FIG. 11. Panel a: schematic showing different layers of the solar cell fabricated starting from the initial gold layer to the completed device; panel b: ten CIS nanocrystal devices on bacterial cellulose substrate; panel c: the strip device with all the solar cells connected in series and voltage being measured with a voltmeter; panel d and panel e: the CIS NC strip device powering a LCD screen in curved position.

FIG. 12. Panels a-d: photographs after CdS deposition on different kinds of paper; panels e-h: SEM images of different kinds of paper after CdS deposition. It can be seen in panel b that the adherence of layers is poor and exposed image paper is visible. A lot of wrinkling is observed in parafilm. SEM images show wrinkling and cracking for wax paper, image paper and parafilm making all of them unsuitable substrates for device fabrication.

FIG. 13. Plots of PCE for CIS NC PVs on bacterial cellulose before ( [symbol] ) and after ( [symbol] ) baking at 200° C. in vacuum and holding for (panel a) 0 minutes, (panel b) 5 minutes, and (panel c) 10 minutes. To get the optimized baking time the devices are heated to 200° C. under vacuum and held for 0 minutes, 5 minutes, and 10 minutes. As seen in the plots, the devices which undergo 5 minute bake perform the best.

FIG. 14. Plot of device characteristics as a function of flex cycles (R=2 mm) for four CIS NC PVs on bacterial cellulose. For a bending radius of 2 mm there is a drop in the performance initially which stabilizes at a later stage.

### Example 2: Applications for Flexible Electronic Devices Including Flexible Photovoltaic Devices

This Example provides an overview of various applications for the flexible electronic devices disclosed herein.

A) Smart labels/stickers. Flexible devices can be used to power smart labels for small-scale digital displays for retail, commercial and industrial application. This enables the elimination of batteries in digital price/info tags in retail stores, as an example. Moreover, these labels can be programmable to change the digital display on a preset or live pattern, eliminating for example, the necessity to change displays manually for retail goods that change price at a high rate, such as gas stations and the price of petrol. A smart label/sticker may correspond to a photovoltaic (PV) paper based smart label in which a flexible PV device is fabricated on a flexible nano-cellulose substrate and an adhesive applied to a surface of the substrate or the PV smart label itself to allow application onto another item.

PV Paper Smart labels can also integrate a variety of sensors into the device, enabling the collection of vital data for marketing research purposes. For example, a smart label might be fitted with a proximity sensor allowing the device to give data on which displayed price point causes the most “customer lingering” around the product; proximity and perhaps heat sensors can also give data on customer “impulse buying”, providing a way for retailers to maximize their product layout for profit.

Smart labels can also be used beyond the application of display, and can be used as a basis to create a “PV powered digital barcode scanner system” which may track more data points on a particular product. On a basic level, this may be an aesthetic consideration, where the label at the very least can enable the barcode to be turned off and on, creating more streamlined aesthetics for marketing. On a more developed level, the digitization and coupling of product barcodes with PV paper technology allows for features such as “warranty alerts” (e.g., when a product is nearing the end of its warranty, the smart label can send an alert to the customer), “product recall alerts” (e.g., when a product is recalled for safety reasons, immediate alerts can be sent to the customer, especially useful for high-risk products such as infant formulas and perishable goods), such as by activating an optical indicator incorporated into the device to allow for determination or identification of these alerts by a consumer. PV paper smart labels can also be used to streamline product exchange and returns by eliminating the need for paper receipts or even credit card tracking by keeping product data in the label itself. For example, wireless data transfer elements, such as an antenna, and data storage devices, such as a flash memory device, may be incorporated into a PV paper smart label.

Product seals: PV paper devices can also be used to create theft-deterrent product seals that ensure that the product has never been opened. A typical product seal functions on a mechanical level, either using adhesion or vacuum/pressure forming processes, both of which can be bypassed easily. A PV paper device seal may be adhered as a seal on a product, using electrical conductivity as the measure of the seal's integrity. If the PV paper seal is altered in any way, the change in conductivity may be registered and the seal deemed “broken”.

B) Smart Packaging. A useful application of PV paper powered smart labels is the ability to track inventory in an immediate and digitized manner. Moreover, PV paper labels can be coupled with devices in the packaging of high-end products that can allow anything from GPS tracking to temperature regulation. GPS tracking of such packages can enable a manner to retrieval and prevention when it comes to the theft of high-cost products which are a target for theft, such as electronic devices, antiquities etc.

Temperature regulation may require a temperature sensor in the packaging of the product to be coupled with these PV paper smart labels, enabling the regulation of products that may be temperature sensitive during the shipping/storing process. Customers concerned about BPA in plastic packaging for instance, may be relieved to find out via the smart label, that the particular bottle of water they have purchased has never reached a temperature of 100 degrees. Such a configuration may be achieved, for example, by including a temperature sensor in or coupled to a PV paper smart label and allowing components in the PV paper smart label to monitor, track, or otherwise determine temperature based on readings from the temperature sensor. An indicator, such as a visual indicator, like a light emitting diode, may be included with or powered by the PV paper smart label and activated or deactivated upon the temperature reaching a particular threshold value.

Many sensors, such as humidity sensors, nitrogen sensors and any industry specific sensor can be coupled and powered with the PV paper smart labels, enabling much more accurate control when bringing a product from manufacturing to the market.

Moreover, packaging developed with PV paper devices can enable packages to track precisely how “rough” they were handled in the delivery process. PV sensors can be printed across the surface of the packages, and, like the “product seal” described above, can track via electrical conductivity, if any surface has been bent or damaged or handled roughly, the shorting of the devices may indicate that. This is especially useful when shipping extremely delicate parcels where vibration and handling may have an effect on the ability for couriers to deliver the product safely. This can also be achieved with force sense resistors, accelerometers, gyroscopes, etc., powered by the PV paper devices, for a more accurate measure. Again, an indicator may be included with or powered by the PV paper device and activated or deactivated upon one or more sensor readings exceeding or falling below a particular threshold value.

C) “Breadcrumbs”: PV paper devices for powering off-grid portable data collection. PV paper devices can provide a cheap alternative to data collection by integrating a disposable, recyclable power source with a variety of sensors used for data collection.

An RFID sensor modified with a PV paper power source for instance, allows a constant broadcast or “ping” to be sent to a centralized data processor, enabling the tracking of animals, plant matter or parts manufacturing in a manner that can be recalled from a remote location. For instance, stickers outfitted with PV paper devices and RFIDs allow a user to venture into uncharted geographical territory, using these devices (referred to in this Example as “breadcrumbs”) to survey the land, chart progress, or even mark out trails and pathways in a discrete or even secretive manner. These devices can also be used to interface with portable electronic devices, enabling developers of location-based virtual applications (such as Pokemon Go!), to create increased interactivity between the virtual and geographic elements of the interface. For instance, in the application of a location-based virtual reality games, users may use these PV paper RFID “breadcrumbs” to trap, control or create structures in the virtual interface of the game.

D) Portable power source for body-integrated devices. Bacterial Nanocellulose is currently used in medical application (in the development of stints) and cosmetics. The material is already conducive to integration with the human body. The ability of the PV paper devices to maintain functionality after flexing, folding and a small degree of stretching, allows for electronic devices including digital displays, processors and sensors, to be used without being tethered to a battery or hardwired power source.

This enables “removable, digital tattoos” that allow the user to mount a flexible digitized screen directly onto the skin with a programmable display. This can be used for cosmetic purposes as well as for tracking bodies post-mortem. Tracking bodies post-mortem can be useful in catastrophic situations where multiple cadavers may need to be tracked and identified, as well as in certain industries, such as medical cadavers or in funeral homes.

Body-Intergraded electronics powered by PV paper devices can go beyond digital display, and can power a variety of sensors now being developed for the human body. Activity trackers can enable users to monitor their health without an external device such as a “Fitbit,” as well as monitor movement for safety and medical reasons. Outfitting an infant with a non-intrusive skin-mounted activity sensor may, for instance, monitor against SIDS. In other cases these sensors could also detect oncoming seizures—and may even be used in conjunction with Vagus Nerve Stimulation devices to prevent seizures from taking place at all. Body-integrated devices such as these can also be used to monitor comatose patients in a less intrusive manner, eliminating the need for sensors to be connected directly to power sources.

Furthermore, the emerging field of Neural Interfaces provides many applications for PV cellulose paper. Currently, neural interfaces such as brain probes for controlling tremors in Parkinson's disease currently do not have a practical power supply solution. Many Neural interfaces that map the electrical activity of individual neurons require a combination of surgically placed batteries that must be replaced in a surgical procedure every 2-3 years, and hardwired power sources (largely used for clinical trials and studies, where the test subject does not necessarily continue the use of the probe outside of the laboratory). In order to develop these probes, which require a large transference of data, for practical, everyday use, a lightweight, body integrated power source is necessary. PV cellulose paper can provide this, at least for low-voltage applications, such as running a wireless data connection from the probe to the computer.

Other implanted devices, such as retinal and cochlear implants currently run off of small replaceable batteries. Having a PV paper option as a power supply can enable implants with less maintenance. Furthermore, a Paper PV powered implant may require a less bulky design (without a battery system), allowing for more comfort and integration into the human body. Other medical implants that benefit from a PV cellulose paper power source are spinal cord stimulators (for pain management). Currently, the field of neural interfaces for the application of pain management constitutes a $4.5 billion industry, and is projected to raise to $14.6 billion by 2024. Most of these implanted devices are limited by their attachment to a bulky or non-portable power source.

Yet another body-integrated device for which PV cellulose paper may have great application is in the field of physical rehabilitation, especially in cases of whole or part paralysis. Commonly, patients that have been paralyzed in some degree need to manage the movement of their body even though they cannot feel perceptually. Pressure sores, for instance, often cause major health issues in the bedridden and in the wheelchair-bound because they cannot feel the pain. Currently there are a few solutions that are integrated into the cushioning of the wheelchair or bed, but very little in terms of a body-integrated warning device. PV paper devices can provide the power for sensors on the body which can help the paralyzed monitor the parts of their body that they cannot feel. Often, patients who are paralyzed are not aware that they may have injured a foot, or an extremity, and have a learning curve at the outset of their condition. Patients need to learn to correlate other symptoms, such as increased heart rate and perspiration to the possibility that they might have injured an extremity with no feeling. Many newly paralyzed patients require a retraining period to look for these warning signs. Body integrated sensors, powered by PV paper devices enable a prosthetic or implanted detection system for identification of injuries in paralyzed patients.

E) PV lightweight paper drones. PV paper devices allow for low-voltage power in a lightweight manner, making it useful for the development of lightweight drones which can function in water. A water-based drone could be made entirely of paper (laminated or coated in fluorocarbon), similar to a paper boat. Low-voltage vibrating motors can be coupled with the PV paper power sources to allow for navigation in the water.

F) PV tape. PV paper devices can be readily integrated into tape rolls, enabling emergency devices for off grid use (e.g., if shipwrecked). Including a roll of PV tape in an emergency kit allows the user to make use of both the adhesive and mechanical qualities of the tape, while also having a power source by which to power a rescue beacon or other such devices.

G) PV wallpaper. PV paper can be developed for architectural applications in both interiors and exteriors, as a low-voltage power-providing wallpaper. This enables a foundation for the development of “smart rooms” which have sensors, displays and processors directly integrated into the architecture. As the PV product can be custom-printed in any configuration, the technology not only allows for a great degree of aesthetic design freedom, but also a way in which to bring multiple data points into a framework of “interactive architecture.” Proximity/temperature sensors, for instance, can track all organisms in any particular building, allowing for faster, more accurate and most importantly more appropriate emergency responses in disaster situations. Government and school buildings, for instance, can give emergency responders accurate locations and counts of civilians when under threat. These sensors can be integrated into other systems in the architecture, such as emergency vaults or safe rooms-in fact entire buildings can be pre-programmed for emergency response protocol, automating evacuation processes and other protocols that are prone to human error.

PV wall paper can also allow for low-voltage charging of electronic devices, eliminating the need for centralized plugs.

Interior PV wallpaper can also be used as a low-voltage power grid for “smart buildings” such as Intelligent Healthcare facilities. Intelligent healthcare facilities are used to measure and diagnose physiological and psychological states using a variety of sensors, such as air quality sensors, motion detectors, light sensors and more. Currently, most of these “smart facilities” use sensors that are not integrated into the infrastructure of the building, requiring independent battery systems to run each sensor. PV wallpaper can be used to supply many more sensors across a larger area, enabling a much greater “resolution” of sensor-based diagnosis. For instance, PV wallpaper can power light and proximity sensors inside a house, giving a measurement of how much natural light an individual is exposed to on a daily basis, or help identify patterns that may lead to the diagnosis of anxiety disorders, compulsive behaviors, depression and productivity. Intelligent Healthcare Facilities such as these have already been able to diagnosis early onset Urinary tract infections based on users' bathroom habits.

Tracking a patient's “wandering” is a great use of these environments, especially in the case of Alzheimer's and Parkinson's disease. The ability for PV wallpaper to provide a battery-less and integrated sensor system for “smart interiors” allows for many more metrics to be possible within a given architectural space. PV wallpaper can also be used to retrofit buildings to allow for this capability, even in off-grid locations.

PV Wallpaper can also allow for architectural structures to self-report on vital statistics in a given building, be it the flow of a population in a museum, or to ensure that maximum occupancy loads for rooms and structures is not exceeded.

Moreover, PV wallpaper can track an individual user throughout a building, enabling a flow of custom AN programming to follow the individual interactively throughout the architecture. For instance, a museum goer could get a customized and tailored art historical audio tour of antiquities triggered by their location and pathway throughout the building simply by walking around.

PV wallpaper can also be developed for exterior use, and given that it can be applied to convex and concave surface curvatures, can provide solar energy on buildings surfaces that are currently un-utilized. The ability to bring a low-voltage interface to the exterior of a building is also valuable for the purpose of creating buildings that gather environmental data on their surroundings. This type of data collection may enable architects and engineers to get direct information on the effect of the environment on the building's longevity and functionality.

Moreover, exterior PV wallpaper allows interactive light displays on the outside of a building, providing a manner in which monitors or screens can be directly installed into the side of a building. If outfitted with cameras, a building with its own social media identity is possible, posting its own pictures and thoughts based on the surrounding environment.

Sensors that measure air quality on the outside of a building for instance, could also provide information relating to risk factors for breathing the air on the top balcony of a high-rise in Shanghai, vs being on a 2nd floor balcony.

H) PV Blinds/window coverings. PV paper devices can be manufactured with a series of folds, (like a venetian blind, or Chinese fan) using knife-pleating methods in roll-to-roll manufacturing. This enables devices that can stretch, flex, be shaped across a complex curvature, and that increase surface area for light absorption. When manufactured in this configuration, PV paper devices can be integrated directly into window coverings, door coverings and awnings which may need to be folded in order to retract. While conventional solar panels require mounting on the rooftop, these PV devices can take advantage of other areas which are prone to a high degree of U exposure.

Moreover, when folded into a conical shape, these devices can be mounted directly only a rooftop, with full coverage and increased surface area.

I) PV “monolithic” infrastructure. PV paper devices can be coupled with inflatable concrete “monolithic” structures in order to produce infrastructure specifically designed to integrate PV paper in an array, or in a building structure. In the construction of a “monolithic” architectural structure, an “airform” is first inflated, creating a general dome shape (shape can be modified). Then foam insulation is sprayed on the inside of the airform while still inflated, and rebar installed and concrete sprayed in the interior. This process creates disaster proof structures that have a history of surviving earthquakes, fires, hurricanes and tornadoes. PV paper devices can be integrated into the outside layer (the “airform”) to enable completely customizable concrete structures to support PV paper arrays as infrastructure. As these structures can be formed in any shape, given that the shape is “inflatable” at the beginning of the process, small round tunnel structures can be formed, enabling a manner in which to build a PV array across many acres, while also providing an interior housing for electronics and maintenance.

J) Space Colony Solar Infrastructure. One of the unique aspects of Bacterial Cellulose as a substrate is that it can be cultivated using nothing more than a wet laboratory set-up, and does not require plant material as pulp; it is generated via the cultivation of bacterial organisms. This fact, coupled with the ability for CIS PVs to be manufactured in a small satellite laboratory, enables the entire process of production for Paper PVs for onsite manufacturing where freight and travel costs are prohibitive. For instance, a production facility for Paper PV devices can be set up on the space station, or in the context of a colony on Mars, and can have the advantage that substrates and heavy materials such as glass do not need to be shipped from Earth to develop solar infrastructure in space. The ability to produce a power solution on location like this enables production facilities where there may be no other supporting infrastructure.

K) PV Power source for paper electrochromic dye-based electronic displays. Electronic displays may be made with nano-cellulose based paper using electrochromic dyes. This enables electronic displays to be fabricated with cellulose paper, producing a tactile, traditional paper-feeling monitor. So far, there have been no appropriate power sources that can integrate into the substrate of these electronic displays. The PV paper devices described herein can serve this purpose and provide the basis for a radical new type of touch-screen interface-one that has the feel of a traditional book.

L) PV Smart Posters. PV Paper can also be used to produce low-voltage powered interactive digital smart posters for the purpose of advertisement, emergency messaging, DIY programmable placards for displaying messages in the context of sports fandom, cue cards for public speaking and political action/protest. PV Paper Posters can be paired with electronic paper displays, interactive sensors, electrochromic dyes, and other devices to produce color-changing, visually responsive posters. As an example, one or more posters or large area substrates may be fabricated and include one or more flexible PV devices, which may be wired to or incorporated with other circuitry or components present on the substrate. Depending on the configuration, flexible display elements, such as incorporating one or more light emitting devices supported by a nano-cellulose substrate, may be incorporated into the device and may be powered by a flexible PV device.

M) PV Textiles. Flexible nano-cellulose paper based PV devices have the ability to be woven directly into textiles, either directly in the weave of the fabric, or as a top coat. As an example, flexible PV devices fabricated on a nano-cellulose substrate may be fabricated into fibers to allow incorporation into a fabric. Alternatively, portions or sections of a textile, fabric, or clothing article, may include a laminated, sewn, or otherwise incorporated nano-cellulose paper based PV device. Functioning as a textile enables PV paper to function in wearable fashion devices, from practical electronic devices (to be integrated into the “Internet of Things”), to aesthetic decorative applications, where electrochromic prints, lights, and iridescence control can be customized. Further applications include large-scale textile uses such as responsive theatrical curtains and sets, flags, banners, advertising, marine-based sails and canopies.

N) Modular PV solar devices. Flexible nano-cellulose paper based PV devices can also be used to create DIY low cost modular solar kits where lightweight PV paper panels can be shipped at low cost to an area of need, to be configured and assembled as modular scalable power grids when combined with readily available materials, such as glass jars, wiring and other objects. The flexible nano-cellulose paper based PV modules can be rolled or wound into rolls for efficient shipping to areas of need. Glass jars or other transparent containers may be useful as storage containers, environmental protection for flexible nano-cellulose paper based PV modules, lensing elements, etc. In use, the flexible nano-cellulose paper based PV modules may be used to line at least a portion of the interior of a glass jar to allow for collection of electrical energy by exposure to light. In some embodiments, battery modules may be included and the flexible nano-cellulose paper based PV modules used to charge the battery during daylight hours. During dark hours, a light emitting device, such as a light emitting diode, may be powered by energy gathered using the flexible nano-cellulose paper based PV modules. Optionally, the jar or a portion thereof may be used to scatter light generated by the light emitting device for more efficient or widespread illumination.

O) Supplemental PV power generation. Flexible nano-cellulose paper based PV devices can also be used to coat the surfaces of other structures for which conventional PV modules are unsuitable. For example, blades of wind turbines, which need to be lightweight, and designed with complex curves—factors that make it difficult for conventional solar panels to be integrated with wind turbines may benefit from having their surfaces lined with flexible nano-cellulose paper based PV devices. Wind turbines may, for example, typically collect energy mostly at night when the wind is high. During the day, such wind turbines may not generate much energy. Outfitting wind turbines with flexible nano-cellulose paper based PV devices to produce a hybrid green energy system is an advantageous way to increase the productivity of wind energy arrays.

P) PV Paper Insect repellant. Flexible nano-cellulose paper based PV devices can also be used as a reusable, humane, fly paper, or flying insect repellant device. By running a low voltage charge through the paper, the light energy harvested by the PV Paper can enable a charge great enough to repel and eliminate bugs should they land on the paper. Such paper can be used as individual devices, similar to the usage of conventional fly paper, or could be used to surface architecture, (window sills, paneling etc. . . . ) to provide a level of security against insects—particularly useful in climates where insect-borne diseases are of concern. As an example, one or more electrical traces can be fabricated across a surface area of a flexible nano-cellulose paper based device and current generated by a PV device incorporated in the flexible nano-cellulose paper based device can be passed through the device.

### X. References

Lim, W.; Douglas, E. A.; Kim, S.-H.; Norton, D. P.; Pearton, S. J.; Ren, F.; Shen, H.; Chang, W. H. High Mobility InGaZnO₄ Thin-Film Transistors on Paper. Appl. Phys. Lett. 2009, 94, 72103.
Eder, F.; Klauk, H.; Halik, M.; Zschieschang, U.; Schmid, G.; Dehm, C. Organic Electronics on Paper. Appl. Phys. Lett. 2004, 84, 2673-2675.
Grau, G.; Kitsomboonloha, R.; Swisher, S. L.; Kang, H.; Subramanian, V. Printed Transistors on Paper: Towards Smart Consumer Product Packaging. Adv. Funct. Mater. 2014, 24, 5067-5074.
Legnani, C.; Vilani, C.; Calil, V. L.; Barud, H. S.; Quirino, W. G.; Achete, C. A.; Ribeiro, S. J. L.; Cremona, M. Bacterial Cellulose Membrane as Flexible Substrate for Organic Light Emitting Devices. Thin Solid Films 2008, 517, 1016-1020.
Ummartyotin, S.; Juntaro, J.; Sain, M.; Manuspiya, H. Development of Transparent Bacterial Cellulose Nanocomposite Film as Substrate for Flexible Organic Light Emitting Diode (OLED) Display. Ind. Crops Prod. 2012, 35, 92-97.
Yoon, B.; Ham, D.-Y.; Yarimaga, O.; An, H.; Lee, C. W.; Kim, J.-M. Inkjet Printing of Conjugated Polymer Precursors on Paper Substrates for Colorimetric Sensing and Flexible Electrothermochromic Display. Adv. Mater. 2011, 23, 5492-5497.
Martinez, A. W.; Phillips, S. T.; Whitesides, G. M.; Carrilho, E. Diagnostics for the Developing World: Microfluidic Paper-Based Analytical Devices. Anal. Chem. 2010, 82, 3-10.
Mazzeo, A. D.; Kalb, W. B.; Chan, L.; Killian, M. G.; Bloch, J.-F.; Mazzeo, B. A.; Whitesides, G. M. Paper-Based, Capacitive Touch Pads. Adv. Mater. 2012, 24, 2850-2856.
Hübler, A.; Trnovec, B.; Zillger, T.; Ali, M.; Wetzold, N.; Mingebach, M.; Wagenpfahl, A.; Deibel, C.; Dyakonov, V. Printed Paper Photovoltaic Cells. Adv. Energy Mater. 2011, 1, 1018-1022.
Barr, M. C.; Rowehl, J. A.; Lunt, R. R.; Xu, J.; Wang, A.; Boyce, C. M.; Im, S. G.; Bulović, V.; Gleason, K. K. Direct Monolithic Integration of Organic Photovoltaic Circuits on Unmodified Paper. Adv. Mater. 2011, 23, 3500-3505.
Wang, B.; Kerr, L. L. Dye Sensitized Solar Cells on Paper Substrates. Sol. Energy Mater. Sol. Cells 2011, 95, 2531-2535.
Kippelen, B.; Brédas, J.-L. Organic Photovoltaics. Energy Environ. Sci. 2009, 2, 251.
Brabec, C. J.; Dyakonov, V.; Parisi, J.; Sariciftci, N. S. Organic Photovoltaics: Concepts and Realization; Springer Science & Business Media, 2013; Vol. 60.
Hillhouse, H. W.; Beard, M. C. Solar Cells from Colloidal Nanocrystals: Fundamentals, Materials, Devices, and Economics. Curr. Opin. Colloid Interface Sci. 2009, 14, 245-259.
Gur, I.; Fromer, N. A.; Geier, M. L.; Alivisatos, A. P. Air-Stable All-Inorganic Nanocrystal Solar Cells Processed from Solution. Science 2005, 310, 462-465.
Fontana, J. D.; De Souza, A. M.; Fontana, C. K.; Torriani, I. L.; Moreschi, J. C.; Gallotti, B. J.; De Souza, S. J.; Narcisco, G. P.; Bichara, J. A.; Farah, L. F. X. Acetobacter Cellulose Pellicle as a Temporary Skin Substitute. Appl. Biochem. Biotechnol. 1990, 24, 253-264.
Czaja, W.; Krystynowicz, A.; Bielecki, S.; Brown Jr., R. M. Microbial Cellulose—the Natural Power to Heal Wounds. Biomaterials 2006, 27, 145-151.
Iguchi, M.; Yamanaka, S.; Budhiono, A. Bacterial Cellulose—a Masterpiece of Nature's Arts. J. Mater. Sci. 2000, 35, 261-270.
Nishi, Y.; Uryu, M.; Yamanaka, S.; Watanabe, K.; Kitamura, N.; Iguchi, M.; Mitsuhashi, S. The Structure and Mechanical Properties of Sheets Prepared from Bacterial Cellulose. J. Mater. Sci. 1990, 25, 2997-3001.
Yoshino, K.; Matsuoka, R.; Nogami, K.; Yamanaka, S.; Watanabe, K.; Takahashi, M.; Honma, M. Graphite Film Prepared by Pyrolysis of Bacterial Cellulose. J. Appl. Phys. 1990, 68, 1720-1725.
Shibazaki, H.; Kuga, S.; Onabe, F.; Usuda, M. Bacterial Cellulose Membrane as Separation Medium. J. Appl. Polym. Sci. 1993, 50, 965-969.
Dubey, V.; Pandey, L. K.; Saxena, C. Pervaporative Separation of Ethanol/water Azeotrope Using a Novel Chitosan-Impregnated Bacterial Cellulose Membrane and Chitosan-poly(vinyl Alcohol) Blends. J. Membr. Sci. 2005, 251, 131-136.
Choi, Y.-J.; Ahn, Y.; Kang, M.-S.; Jun, H.-K.; Kim, I. S.; Moon, S.-H. Preparation and Characterization of Acrylic Acid-Treated Bacterial Cellulose Cation-Exchange Membrane. J. Chem. Technol. Biotechnol. 2004, 79, 79-84.
Dubey, V.; Saxena, C.; Singh, L.; Ramana, K. V.; Chauhan, R. S. Pervaporation of Binary Water-ethanol Mixtures through Bacterial Cellulose Membrane. Sep. Purif. Technol. 2002, 27, 163-171.
Shah, J.; Jr, R. M. B. Towards Electronic Paper Displays Made from Microbial Cellulose. Appl. Microbiol. Biotechnol. 2004, 66, 352-355.
Legnani, C.; Vilani, C.; Calil, V. L.; Barud, H. S.; Quirino, W. G.; Achete, C. A.; Ribeiro, S. J. L.; Cremona, M. Bacterial Cellulose Membrane as Flexible Substrate for Organic Light Emitting Devices. Thin Solid Films 2008, 517, 1016-1020.
Yano, H.; Sugiyama, J.; Nakagaito, A. N.; Nogi, M.; Matsuura, T.; Hikita, M.; Handa, K. Optically Transparent Composites Reinforced with Networks of Bacterial Nanofibers. Adv. Mater. 2005, 17, 153-155.
Kessler, F.; Herz, K.; Powalla, M.; Hartmann, M.; Schmidt, M.; Jasenek, A.; Schock, H. W. Flexible and monolithically integrated CIGS-Modules. MRS Proceedings, 2001, 668, H3.6, doi: 10.1557/PROC-668-H3.6.
Kapur, V. K.; Bansal, A.; Le, P.; Asensio, O. I. Non-Vacuum Processing of CuI_{1-X}Ga_{X}Se₂ Solar Cells on Rigid and Flexible Substrates Using Nanoparticle Precursor Inks. Thin Solid Films 2003, 431, 53-57.
Reinhard, P.; Chirila, A.; Blosch, P.; Pianezzi, F.; Nishiwaki, S.; Buechelers, S.; Tiwari, A. N. Review of Progress toward 20% Efficiency Flexible CIGS Solar Cells and Manufacturing Issues of Solar Modules. Photovolt. IEEE J. Of 2013, 3, 572-580.
Herrmann, D.; Kessler, F.; Herz, K.; Powalla, M.; Schulz, A.; Schneider, J.; Schumacher, U. High-Performance Barrier Layers for Flexible CIGS Thin-Film Solar Cells on Metal Foils. In MRS Proceedings; Cambridge Univ Press, 2003; Vol. 763, pp. B6-10.
Chiril\check{a}, A.; Buecheler, S.; Pianezzi, F.; Bloesch, P.; Gretener, C.; Uhl, A. R.; Fella, C.; Kranz, L.; Perrenoud, J.; Seyrling, S.; et al. Highly Efficient Cu (In, Ga) Se2 Solar Cells Grown on Flexible Polymer Films. Nat. Mater. 2011, 10, 857-861.
Akhavan, V. A.; Panthani, M. G.; Goodfellow, B. W.; Reid, D. K.; Korgel, B. A. Thickness-Limited Performance of CuInSe₂ Nanocrystal Photovoltaic Devices. Opt. Express 2010, 18, A411.
Panthani, M. G.; Stolle, C. J.; Reid, D. K.; Rhee, D. J.; Harvey, T. B.; Akhavan, V. A.; Yu, Y.; Korgel, B. A. CuInSe₂ Quantum Dot Solar Cells with High Open-Circuit Voltage. J. Phys. Chem. Lett. 2013, 4, 2030-2034.
Tang, R.; Huang, H.; Tu, H.; Liang, H.; Liang, M.; Song, Z.; Xu, Y.; Jiang, H.; Yu, H. Origami-Enabled Deformable Silicon Solar Cells. Appl. Phys. Lett. 2014, 104, 83501.
Lamoureux, A.; Lee, K.; Shlian, M.; Forrest, S. R.; Shtein, M. Dynamic Kirigami Structures for Integrated Solar Tracking. Nat. Commun. 2015, 6.
Brown, R. M.; Willison, J. H.; Richardson, C. L. Cellulose Biosynthesis in Acetobacter Xylinum: Visualization of the Site of Synthesis and Direct Measurement of the in Vivo Process. Proc. Natl. Acad. Sci. U.S.A. 1976, 73, 4565-4569.
U.S. Pat. No. 6,537,845.
U.S. Pat. No. 4,942,128.
U.S. Pat. No. 8,951,551.
U.S. Patent Application Publication No. 2008/0297878.
U.S. Patent Application Publication No. 2005/0079386.
U.S. Patent Application Publication No. 2011/0056564.
Leonat, L.; White, M. S.; Glowacki, E. D.; Scharber, M. C.; Zillger, T.; Rühling, J.; Hübler, A.; Sariciftci, N. S. 4% Efficient Polymer Solar Cells on Paper Substrates. J. Phys. Chem. C 2014, 118, 16813-16817.
Fatemi, N. S.; Pollard, H. E.; Hou, H. Q.; Sharps, P. R. Solar Array Trades between Very High-Efficiency Multi-Junction and Si Space Solar Cells. In Conference Record of the Twenty-Eighth IEEE Photovoltaic Specialists Conference, 2000; 2000; pp. 1083-1086.
Gaynor, W.; Burkhard, G. F.; McGehee, M. D.; Peumans, P. Smooth Nanowire/Polymer Composite Transparent Electrodes. Adv. Mater. 2011, 23, 2905-2910.
Kumar, A.; Zhou, C. The Race To Replace Tin-Doped Indium Oxide: Which Material Will Win? ACS Nano 2010, 4, 11-14.
Wu, H.; Hu, L.; Rowell, M. W.; Kong, D.; Cha, J. J.; McDonough, J. R.; Zhu, J.; Yang, Y.; McGehee, M. D.; Cui, Y. Electrospun Metal Nanofiber Webs as High-Performance Transparent Electrode. Nano Lett. 2010, 10, 4242-4248.
Rowell, M. W.; Topinka, M. A.; McGehee, M. D.; Prall, H.-J.; Dennler, G.; Sariciftci, N. S.; Hu, L.; Gruner, G. Organic Solar Cells with Carbon Nanotube Network Electrodes. Appl. Phys. Lett. 2006, 88, 233506.
Lin, Q.; Huang, H.; Jing, Y.; Fu, H.; Chang, P.; Li, D.; Yao, Y.; Fan, Z. Flexible Photovoltaic Technologies. I Mater. Chem. C 2014, 2, 1233.
Kaltenbrunner, M.; White, M. S.; Glowacki, E. D.; Sekitani, T.; Someya, T.; Sariciftci, N. S.; Bauer, S. Ultrathin and Lightweight Organic Solar Cells with High Flexibility. Nat. Commun. 2012, 3, 770.
Seshan, C. Cell Efficiency Dependence on Solar Incidence Angle. In 2010 35th IEEE Photovoltaic Specialists Conference (PVSC); 2010; pp. 002102-002105.
Tvingstedt, K.; Andersson, V.; Zhang, F.; Inganäs, O. Folded Reflective Tandem Polymer Solar Cell Doubles Efficiency. Appl. Phys. Lett. 2007, 91, 123514.
Zhou, Y.; Zhang, F.; Tvingstedt, K.; Tian, W.; Inganas, O. Multifolded Polymer Solar Cells on Flexible Substrates. Appl. Phys. Lett. 2008, 93, 33302.
Vosgueritchian, M.; Lipomi, D. J.; Bao, Z. Highly Conductive and Transparent PEDOT:PSS Films with a Fluorosurfactant for Stretchable and Flexible Transparent Electrodes. Adv. Funct. Mater. 2012, 22, 421-428.
Bae, S.; Kim, H.; Lee, Y.; Xu, X.; Park, J.-S.; Zheng, Y.; Balakrishnan, J.; Lei, T.; Ri Kim, H.; Song, Y. I.; et al. Roll-to-Roll Production of 30-Inch Graphene Films for Transparent Electrodes. Nat. Nanotechnol. 2010, 5, 574-578.
Li, X.; Zhu, Y.; Cai, W.; Borysiak, M.; Han, B.; Chen, D.; Piner, R. D.; Colombo, L.; Ruoff, R. S. Transfer of Large-Area Graphene Films for High-Performance Transparent Conductive Electrodes. Nano Lett. 2009, 9, 4359-4363.
Rathmell, A. R.; Wiley, B. J. The Synthesis and Coating of Long, Thin Copper Nanowires to Make Flexible, Transparent Conducting Films on Plastic Substrates. Adv. Mater. 2011, 23, 4798-4803.
De, S.; Higgins, T. M.; Lyons, P. E.; Doherty, E. M.; Nirmalraj, P. N.; Blau, W. J.; Boland, J. J.; Coleman, J. N. Silver Nanowire Networks as Flexible, Transparent, Conducting Films: Extremely High DC to Optical Conductivity Ratios. ACS Nano 2009, 3, 1767-1774.

### XI. Statements Regarding Incorporation by Reference and Variations

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”.

Every formulation or combination of components described or exemplified can be used to practice the invention, unless otherwise stated. Specific names of materials are intended to be exemplary, as it is known that one of ordinary skill in the art can name the same material differently. One of ordinary skill in the art will appreciate that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims. In addition, it will be appreciated that any and all embodiments and aspects disclosed herein are not intended to be and are not mutually exclusive. In other words, embodiments and aspects disclosed herein may be combined and aspects or features of different embodiments or aspects may be combined with other embodiments or aspects, without limitation.

What is claimed is:

1. A method of making a flexible optoelectronic device, comprising:
providing a flexible substrate comprising cellulose nano-fibers having average diameters between about 50 nm and about 150 nm; and
depositing one or more layers over the flexible substrate, wherein the one or more layers include at least a crystalline inorganic semiconductor material, and wherein the crystalline inorganic semiconductor material is deposited over the flexible substrate using nanocrystals in a solution processing method.

2. The method of claim 1, wherein depositing the one or more layers comprises:
depositing a first conductor layer over the flexible substrate;
depositing the crystalline inorganic semiconductor material over the first metal layer to form a first semiconductor layer;
depositing a second semiconductor material over the first semiconductor layer to form a second semiconductor layer, wherein one of the crystalline inorganic semiconductor material or the second semiconductor material comprises an n-type semiconductor, and wherein one of the crystalline semiconductor material or the second semiconductor material comprises a p-type semiconductor; and
depositing a second optically transparent conductor layer over the flexible substrate.

3. The method of claim 2, wherein:
depositing the first conductor layer comprises using a first dry deposition processing method;
depositing the crystalline inorganic semiconductor material comprises depositing a suspension of nanoparticles of the crystalline inorganic semiconductor material over the first conductor layer and allowing solvent of the suspension to evaporate;
depositing the second semiconductor layer comprises using at least one of a second solution processing method or a second dry deposition processing method; or
depositing the second conductor layer comprises using a third dry deposition process.

4. The method of claim 2, wherein at least one of the first dry deposition processing method, the second dry deposition processing method, or the third dry deposition processing method is selected from the group consisting of a physical vapor deposition method, an evaporation deposition method, thermal evaporation, a sputtering deposition method, radio frequency sputtering, and any combination of these.

5. The method of claim 1, wherein the solution processing method comprises one or more of spray coating a suspension of the nanocrystals, drop casting a suspension of the nanocrystals, spin coating a suspension of the nanocrystals, inkjet printing a suspension of the nanocrystals, or spreading a suspension of the nanocrystals using a doctor blade.

6. The method of claim 2, wherein the second solution processing method comprises a chemical bath deposition process.

7. The method of claim 2, wherein the flexible device component is a photovoltaic cell, wherein the first conductor layer comprises a metal, wherein the crystalline inorganic semiconductor material comprises a CuInSe₂ nanocrystal film, wherein the second semiconductor material comprises CdS and/or ZnO, and wherein the second conductor layer comprises ITO.

8. The method of claim 7, wherein the photovoltaic cell exhibits a power conversion efficiency of at least 1%.

9. The method of claim 1, wherein providing the flexible substrate comprises:
growing a culture of a cellulose producing bacteria;
harvesting the cellulose from the culture;
pressing the cellulose to form a sheet of bacterial cellulose paper; and
drying the sheet of bacterial cellulose paper.

10. The method of claim 1, wherein the flexible substrate exhibits a surface roughness of from 30 nm to 120 nm

11. The method of claim 1, wherein the layers are free of cracks that cause failure or short circuiting of the electronic device component.

12. The method of claim 1, wherein the one or more flexible electronic devices correspond to a plurality of flexible photovoltaic cells, the method further comprising:
arranging the plurality of flexible photovoltaic cells in a series configuration;
electrically connecting the plurality of flexible photovoltaic cells to a display.

13. A method of operating a flexible electronic device, comprising:
providing the flexible electronic device, wherein the flexible electronic device comprises:
a flexible substrate comprising paper including cellulose nano-fibers having average diameters between about 50 nm and about 150 nm; and
a flexible electronic device component supported by the flexible substrate, wherein the flexible electronic device component comprises a crystalline inorganic semiconductor material; and
providing current or voltage to the flexible electronic device component or generating current or voltage using the flexible electronic device component.

14. The method of claim 13, further comprising:
bending the flexible substrate to a radius of curvature of between 3 mm and 5 mm.

15. The method of claim 13, further comprising:
stretching the flexible substrate along a lateral direction to between 105% and 150% of an unstretched lateral size.

16. The method of claim 13, further comprising:
folding the flexible substrate; and
unfolding the flexible substrate.

17. The method of claim 13, wherein the flexible electronic device comprises a label, a sticker, a sensor, a body-integrated device, a drone, a photovoltaic tape, a photovoltaic wallpaper, a photovoltaic window covering, a man-made structure, an electronic display, and any combination of these.

18. The method of claim 13, wherein the flexible device component is a photovoltaic cell comprising:
a first conductor layer over the flexible substrate;
a first semiconductor layer over the first conductor, the first semiconductor layer comprising the crystalline inorganic semiconductor material;
a second semiconductor layer over the first semiconductor layer; and
a second conductor layer over the second semiconductor layer.

19. The method of claim 18, wherein the photovoltaic cell exhibits a power conversion efficiency of at least 1%.

20. The method of claim 18, wherein the flexible device component comprises a plurality of additional photovoltaic cells arranged a series configuration with the photovoltaic cell and electrically to a display to provide a voltage to the display.
